(19) United States
(12) Patent Application Publication
(10) Pub. No.: US 2009/0327170 A1
(43) Pub. Date: Dec. 31, 2009

(54) Methods of Clustering Gene and Protein Sequences

(76) Inventors: Claudio Donati, Poggibonsi (IT); Duccio Medini, Perugia (IT); Antonello Covacci, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/086,717

(22) PCT Filed: Dec. 19, 2006
(86) PCT No.: PCT/IB2006/003901

§ 371 Date: Apr. 27, 2009

Related U.S. Application Data
(60) Provisional application No. 60/751,804, filed on Dec. 19, 2005.
(60) Provisional application No. 60/857,297, filed on Nov. 6, 2006.

(51) Int. Cl. G06F 15/18; C07K 14/00; C07K 16/18; C12N 15/11; A61K 39/395; A61K 39/00; A61K 31/7088; A61K 38/16

(52) U.S. Cl. 706/12; 530/350; 530/387.9; 536/23.1; 424/139.1; 424/185.1; 514/44 R; 514/12; 706/54

(57) ABSTRACT

The invention relates to methods for clustering gene and protein sequences. In particular, it involves generation of networks of sequences where the interconnections are based upon a measure of similarity. The invention also provides methods of optimizing and improving the networks by re-wiring of the network based upon overlap of the nearest neighbors of given pairs of nodes. The invention further provides methods of identifying clusters of sequences within the networks and the optimized networks based upon the topology of the network. The clusters identified represent groups of sequences that are related by function and/or evolution. The invention has particular applicability in annotation of sequences in databases and identification of functional homologs which can be very useful for novel therapeutic and diagnostic targets based upon such targets belonging to a cluster or family that contains a known sequence such as a diagnostic sequence, antigen or other therapeutic target.

### FIELD OF THE INVENTION

The present invention relates to the fields of bioinformatics. In particular, the present invention relates to identifying families or clusters of related sequences within datasets of protein and/or nucleic acid sequences. In addition, the present invention relates to proteins and nucleic acid sequences identified by the present methods and methods for use of the proteins and nucleic acid sequences for diagnosis, treatment and prevention of pathogen infection and methods of generating compositions for such uses.

### BACKGROUND OF THE INVENTION

Starting from the pioneering works of M. O. Dayhoff on bio-molecular evolution (1, 2), the classification of proteins into families with common ancestors has been one of the major tasks of bioinformatics (2, 3). Traditionally, this classification has involved use of computer programs such as blast to perform pair-wise comparisons of the proteins at the level of the primary sequence. Such alignments may be used to generate family trees based upon the relative similarities among the sequences being compared. More advanced algorithms are available that use sequence alignments to construct phylogenetic trees that are optimized based upon parsimony, distance, or maximum likelihood criteria.

In recent years, with the extraordinary increase of genomic data, the complexity of this task has grown enormously. In parallel, the importance of this type of classification has also been increasingly been recognized in understanding the processes leading to species formation and diversification. The availability of complete genomes has shown that the transmission of genetic material between different organisms, whose importance had already been recognized in the field of bacterial pathogenesis (4), is a frequent occurrence, and has probably shaped the evolution of many living organisms (5). It has been proposed that the concept of the phylogenetic tree of the living organisms should be instead be replaced by a phylogenetic network, where connections between different clades occur due to events of horizontal gene transfer (6). The non-trivial relationships connecting separated branches of the tree of life are more easily detectable once each gene product encoded in the different genomes has been classified in a protein family. Each genome is reduced to a list of protein families, allowing one to identify the existence of conserved functions, pathways or organelles in different species. In addition, since the classification highlights evolutionary relationships, correlated evolutionary history of different systems or system components is easily detectable.

There are a number of examples of using networks to describe a wide rage of systems in biology (29, 30, 31, 32) and in the social sciences (33, 34, 35, 36). Despite the networks describing disparate systems, they all share certain features including a power law decay of the distribution of the number of links departing from a node and a high degree of compactness on a local scale. The problem of partitioning a network into a set of communities has been studied in detail in the context of the social sciences, and several algorithms have been proposed, which quantify the probability that a particular link connects different communities (19). However, these algorithms are based on global properties of the network, and require an evaluation of all the paths that use a certain link. This feature, together with the iterative nature of these methods, makes it unfeasible to apply them to large datasets. Given the increasing numbers of organisms whose entire genome has been sequenced, the amount of data available for comparison of protein and nucleic acid sequences has expanded dramatically. The sheer amount of data precludes the use of partitioning algorithms given that partitioning algorithms require the complete enumeration of all possible classifications (28) or the recursing elimination of weak links. Thus, there is a need for robust methods of identifying families of proteins that may be applied to large networks, e.g., generated using sequences from multiple genomes, and that are not as computationally intensive as current methods.

### SUMMARY

The present invention addresses these needs by providing methods for clustering proteins that are both more robust than traditional methods using phylogenetic trees and less computationally intensive than traditional network clustering methods. The methods of the present invention described herein can leverage the topological properties of sequence similarity networks, reducing considerably the computational load associated with the partitioning, rendering them applicable to the growing protein and nucleic acid sequence databases.

One aspect of the present invention provides methods for generating sequence similarity networks that have one or more sequence similarity families from a dataset of sequences or otherwise partition such sequence similarity networks into one or more sequence similarity families. In some embodiments, the sequence similarity networks are generated from the dataset of sequences where each node in the sequence similarity network represents a sequence from the dataset and each pair of nodes is connected by a link if a sequence similarity criterion is met for the pair of nodes. In certain embodiments, the sequence similarity criterion is met when the sequence similarity index for a pair of sequences indicates similarity more significant than a sequence similarity threshold. In preferred embodiments, the sequence similarity indices will be E-values and for such embodiments, the preferred sequence similarity thresholds are about 1, about 10⁻¹, about 10⁻², about 10⁻³, about 10⁻⁴, about 10⁻⁵, about 10⁻⁶, about 10⁻⁷, about 10⁻⁸, about 10⁻¹⁰, about 10⁻¹⁵, about 10⁻²⁰, about 10⁻³⁰, or in the range of about 10⁻¹ to about 10⁻⁴⁰, about 10⁻⁵ to about 10⁻³⁰. In some embodiments, the sequence similarity indices will be percent identity and the preferred sequence similarity thresholds are about 35%, about 40%, about 45%, about 50%, about 60%, about 70%, about 75%, about 80%, about 85%, about 90%, about 95%, or in the range of about 35% to about 95%, or about 45% to about 85% identity.

In some embodiments, the dataset of sequences will have at least about 100, at least about 1000, at least about 10,000, at least about 100,000, or at least about 1,000,000 sequences. In preferred embodiments, the sequences may be nucleic acid sequences including by way of example gene sequences, promoter sequences, cDNA sequencing, protein coding sequences, protein domain coding sequences, exon sequences, intron sequences, In other preferred embodiments, the sequences may be protein sequences including entire protein sequences, fragments of protein sequences, protein domain sequences, and sequences of proteins corresponding to exons.

In preferred embodiments, the sequence similarity network will be rewired or partitioned into sequence similarity families by applying an overlap criterion to at least one pair of nodes. In certain embodiments, the overlap criterion will be applied to at least 20%, at least 40%, at least 60%, at least 80% or all of the pairs of nodes. In other embodiments, the overlap criterion will only be applied where both nodes have less than a threshold number of links. In preferred embodiments, the rewiring or partitioning will include removal of links between pairs of nodes where the overlap is not met. In preferred embodiments, the links removed will include at least fifty percent false links, at least seventy percent false links, at least eighty percent false links, at least ninety percent false links, or at least ninety-five percent false links. In preferred embodiments, the rewiring or partitioning will include addition of links between pairs of nodes where the overlap is met. In preferred embodiments, the links added will include fewer than sixty percent false links, fewer than fifty percent false links, fewer than forty false links, fewer than thirty percent false links, or fewer than twenty percent false links. One of skill in the art will recognize that any criterion may be reversed and therefore the rewiring or partitioning overlap criterion may require removal of links meeting the overlap criterion and/or adding links not meeting the overlap criterion.

In some embodiments, the overlap criterion will be met when an overlap coefficient for a pair of sequences is greater than or equal to an overlap threshold. In certain aspects the overlap threshold may determined by calculating the average connectivity coefficient for each sequence similarity network generated by rewiring or partitioning the sequence similarity network for a set of overlap thresholds and selecting an overlap threshold from the set of overlap thresholds that yields a modularity coefficient of at least about 0.3. In preferred embodiments, the selected overlap threshold will yield a modularity coefficient of at least about 0.4, at least about 0.5, at least about 0.6, at least about 0.65, or at least about 0.7. In some embodiments overlap threshold selected will yield the highest modularity coefficient. In certain embodiments, the overlap threshold will be between about 0.2 and about 0.9, between about 0.3 and about 0.8, or between about 0.4 and about 0.6. In preferred embodiments, the overlap threshold will be about 0.5.

Another aspect of the present invention includes use of the methods to the sequence similarity family that includes a protein of interest. In certain embodiments sequence of interest is an antigenic protein sequence, an antibody therapeutic target protein sequence, or a small molecule therapeutic target protein sequence. In preferred embodiments, at least one other sequences in the same sequence similarity family will be selected as a potential antigenic protein sequence, a potential antibody therapeutic target protein sequence, or a potential small molecule therapeutic target protein sequence

Another aspect of the present invention include annotating sequences within a dataset of sequences using any of the aspects and embodiments of the present invention to rewire or partition a sequence similarity network to produce sequence similarity families. In various embodiments, the dataset of sequences will include one or more, two or more, ten or more, one hundred or more, one thousand or more, or ten thousand or more annotated sequences (which may be fully or only partly annotated) and one or more, two or more, ten or more, one hundred or more, one thousand or more, or ten thousand or more unannotated or partly annotated sequences. In preferred embodiments, the unannotated or partly annotated sequences will be annotated by adding the annotation from any annotated sequences in the same sequence similarity family. In some embodiments, the annotations will be improved by comparing all the annotations of the annotated sequences within a sequence similarity family and removing the annotations that represent a minority of the annotations.

Another aspect of the present invention include identifying an evolutionarily-related families of sequences within a dataset of sequences using any of the aspects and embodiments of the present invention to rewire or partition a sequence similarity network to produce sequence similarity families. In various embodiments, the dataset of sequences will include one or more, two or more, ten or more, one hundred or more, one thousand or more, or ten thousand or more evolutionarily-related sequences. In preferred embodiments, rewiring or partitioning will remove at least one sequence from the sequence similarity family that is not evolutionarily related to the sequences in the sequence similarity family, but has greater homology at the primary sequence level to at least one sequence in the sequence similarity family than between at least one pair of sequences in the sequence similarity family.

Any and all of the aspects of the present invention may be implemented though computerized systems. A preferred aspect is computer-readable media that has computer-executable instructions for performing any of the methods of the present invention including without limitation generating or partition a sequence similarity network that has one or more sequence similarity families from a dataset of sequences and annotating sequences within a dataset of sequences (including all embodiments discussed above and throughout the specification). Another preferred aspect includes computerized systems for performing any of the methods of the present invention including without limitation generating or partitioning a sequence similarity network that has one or more sequence similarity families from a dataset of sequences and annotating sequences within a dataset of sequences (including all embodiments discussed above and throughout the specification). Yet another aspect includes computerized systems comprising a computer-readable medium containing a sequence similarity network comprising one or more sequence similarity families generated, partitioned and/or annotated using any of the methods of the present invention.

### BRIEF DESCRIPTION OF THE FIGURES

The figures provided are as follows:

FIG. 1: Shows a graph comparing the fraction n_{G} of nodes in the largest connected component of the sequence similarity network in the Examples at different cut-offs of ε.

FIG. 2: (A) Shows the probability distribution of the node compactness index ηᵢ for ε=10⁻¹⁰⁰ (open circles) and ε=10⁻⁵ (full circles). (B) Shows the Probability distribution of the node clustering index Cᵢ for four values of ε with the average clustering index C shown as a function of ε in the inset graph.

FIG. 3: Shows a graph of the compactness index η at various cut-offs of θ. The inset shows a graph of the modularity measure Q at various cut-offs of θ.

FIG. 4: (A) Shows a network representation of the SctJ sequence similarity family before re-wiring based upon the overlap (absolute cut-off for ε=10⁻⁵ with gradations in ε shown color in according to the ruler on the right). Two subgroups are visible within the central cluster that correspond to the YscJ (TTSS) and FliF (flagellar) proteins. The outliers showing in blue connect the family to the giant component. After re-wiring with the overlap procedure, false links to the outliers are removed and the SctJ proteins all fall within a single sequence similarity family (shown with the circle). The network representation was generated with the aid of the Tulip 2.0.0 graphic library (available on the Internet at labri.fr under the directory perso/auber/projects/tulip/). (B) Shows the maximum likelihood phylogenetic tree of the proteins included in the SctJ family. The two subgroups in the network representation in (A) correspond to the two distinct evolutionary clades. The organism and group names in the TTSS clade refer to the TTSS classifications shown in FIG. 6.

FIG. 5: Shows the maximum likelihood phylogenetic tree for the 33 proteins classified in the 3 sequence similarity families associated with the functional group VirB. The sequence similarity families identified in the Examples are enclosed in circles. The color coding matches the color coding in FIG. 6. The ruler bar shows the number of Point Accepted Mutations.

FIG. 6: Shows the sequence similarity families identified in the Examples for the two different systems (A: TTSS; B: TFSS). Protein functional groups are ordered by column. The colors identify different sequence similarity families. White indicates a lack of a corresponding protein in the organism (or plasmid); grey indicates conserved proteins. The two external reference systems are indicated in bold (E. coli flagellar apparatus for TTSS and a Tra/Trb conjugative system for TFSS). The dendrograms represent a hierarchical agglomerative clustering of the data that highlights the presence of five and fore major groups (roman numerals) in TTSS and TFSS, respectively.

FIG. 7: Shows a graph of the compactness index q for various cut-offs of 6 for the complete network (full circles) and the network without the giant component (open circles).

### DETAILED DESCRIPTION OF THE PREFERRED EMBODIMENTS

The present invention is directed to methods and compositions for defining families or clusters of similar sequences. The present invention is particularly useful for defining families or clusters that have an evolutionary and/or functional relationship. The families or clusters may be defined by topological evaluation and partitioning of sequence similarity networks. Sequence similarity networks are formed based upon the similarity relationships between sequences that may be inferred from the similarity between the sequences at the primary level. Due to the transitivity of the similarity relationships, an ideal sequence similarity network, i.e., where only truly similar sequences are connected, will be composed of sets of disconnected sub-networks, where all pairs of similar sequences are connected by a link, and non-similar sequences belong to distinct sub-networks. In preferred embodiments, the sequence similarity network is rewired by an overlap procedure that add links between sequences in the network that share the minimum overlap in nearest neighbors and removes links between sequences that do not share a certain minimum overlap. In more preferred embodiments, this rewiring procedure will preferentially remove at least about fifty percent false links, at least seventy percent false links, at least eighty percent false links, at least ninety percent false links, or at least ninety-five percent false links and/or add fewer than sixty percent false links, fewer than fifty percent false links, fewer than forty false links, fewer than thirty percent false links, or fewer than twenty percent false links false links, thus improving the quality of the sequence similarity network.

In most preferred embodiments, each of these clusters of sequences or sequence similarity families, being formed only of similar sequences, provide a family of homologous proteins or nucleic acids. When homology is inferred only from sequence similarity, false or missing links can alter the structure of the network, making it difficult to define the boundaries of the different protein or nucleic acid families. Nevertheless, it is still possible to recognize that the density of links is higher in some regions of the network than in others, and protein or nucleic acid families can be identified within these compact regions. The present invention uses the topological properties of sequence similarity networks to define a new similarity measure among the sequences that allows one to better identify densely connected regions, and to classify large sets of protein or nucleic acids into families. The present invention also provides methods of rewiring the networks based upon the overlap in nearest neighbors between pairs of sequences in the network. Such rewiring improves the quality of the sequence similarity network, e.g., removing false links so that the sequences may be divided into distinct clusters or sequence similarity families within the network.

Set of Sequences to be Clustered

The methods of the present invention may be applied to any database of protein and/or nucleic acid sequences where there are sequences within the database that have some degree of similarity and may include dissimilar sequences as well. In some preferred embodiments, the database will include protein sequences. Such protein sequences can be entire protein sequences or smaller fragments of proteins, such as a database that has proteins divided by domains. In some embodiments, the database can comprise nucleic acid sequences. The sequences can be entire genes (i.e., promoters, non-transcribed and non-translated regions as well as coding regions), transcribed regions such as entire cDNA, coding regions within cDNA, and promoters and/or enhancers of a gene. Similarly, the coding regions of cDNAs can be broken into smaller fragments such as exons or fragments that code for individual protein domains.

Given the robustness of the methods of the present invention, the databases will preferably include entire genomes of as many organisms as reasonable for the desired comparison. However, the methods can be equally applied to smaller databases such as databases of genomes from particular groups of organisms such as prokaryotes, eubacteria, archaea, eukaryotes, plants, animal, fungi, mammals, etc. In addition, the databases may comprise incomplete genomes, portions of genomes, plasmids, organelle genomes, and viral genomes.

Similarity Indices

In some embodiments, the sequence similarity networks of the present invention are generated using a similarity index. The similarity index εᵢⱼ is a numerical value that represents the similarity between a pair of sequences (i, j) at the primary level. A wide range of programs are available for alignment of sequences at the primary level. Examples of such programs include: blastn, blastp, fasta, psi-blast, pileup, etc. Each of the programs typically output one or more measures of similarity between sequences. Examples of such measures include percent identity, percent similarity, E-value, and the negative log-likelihood minus NULL model (NLL-NULL, or log-odds) scores. One of skill in the art will recognize other such measures useful in the present invention. A preferred similarity index is the E-value, which represents an estimated number of alignments of equal or better quality that could be found by pure chance in a database. The NLL-NULL value may be calculated by the SAM (Sequence Alignment and Modeling) suite (available at cse.ucsc.edu in the folder research/compbio/sam.html). Percent identity is the percentage of identical amino acids shared in an alignment of a pair of sequences (which may be modified to include penalties for gaps in the alignment, etc.). Percent similarity is the percent of the homologous amino acids shared in an alignment of a pair of sequence (which again may be modified to include gaps in the alignment, etc.).

The sequence similarity index is generally a measure of homology between sequences. Such homology can be determined using standard techniques known in the art, including, but not limited to, the local homology algorithm Smith & Waterman (37), by the homology alignment algorithm of Needleman & Wunsch (38), by the search for similarity method of Pearson & Lipman, (39), by computerized implementations of these algorithms (GAP, BESTFIT, FASTA, and TFASTA in the Wisconsin Genetics Software Package, Genetics Computer Group, 575 Science Drive, Madison, Wis.), or the Best Fit sequence program described by Devereux et al. (40), preferably using the default settings, or by inspection.

Another example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng & Doolittle (41); the method is similar to that described by Higgins & Sharp (42). Useful PILEUP parameters include a default gap weight of 3.00, a default gap length weight of 0.10, and weighted end gaps.

Another example of a useful algorithm is the BLAST (Basic Local Alignment Search Tool) algorithm, described in Altschul et al. (43) and Karlin et al. (44). A particularly useful BLAST program is the WU-BLAST-2 program which was obtained from Altschul et al. (45); available on the web at blast.wustl.edu. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Generation of Networks

The sequence similarity network can be generated by applying a sequence similarity criterion to the dataset of sequences whereby similar sequences will be connected by a link or edge, preferably in a pairwise fashion. The preferred sequence similarity criterion is applied by generating a network where the sequences are the nodes and any pair of nodes i, j are connected by an undirected edge if and only if the εᵢⱼ is smaller (or larger depending upon the nature of the similarity index) than a given threshold ε. In preferred embodiments, no distinction is made between links with different values of εᵢⱼ. While the number of vertexes N in the network (the network size) is fixed by the number of sequences in the dataset, the number of links, and consequently the structure of the network, depends on the cut-off adopted.

The maximum number of links allowed by the network size will be (N(N−1))/2. With increasingly stringent cutoff conditions, the network will have fewer links. Various methods are available to optimize the cutoff to be used in generating the network. An ideal cutoff is one which minimizes the number of false links while maximizing the number of correct links.

The network connectivity is a useful measure for evaluation of the topology of a network and therefore its quality. Connectivity on a local scale can be evaluated using the clustering index Cᵢ, which is defined as (22):

Cᵢ=(2Eᵢ)/(kᵢ(kᵢ−1))

where Eᵢ is the number of edges among the kᵢ nearest neighbors. If the i-th vertex and its nearest neighbors form a clique, Cᵢ=1; if the 1-th vertex is at the center of a star-like topology, Cᵢ is 0. The network clustering index C is the average of the node clustering index over the whole network is:

C=(1/N)ΣCᵢ

where N is the number of nodes in the network. An example of an alternative measure of connectivity is where Cᵢ is equal to the fraction of the number of links between neighbors of a node and the total possible number of links between neighbors of the node (49).

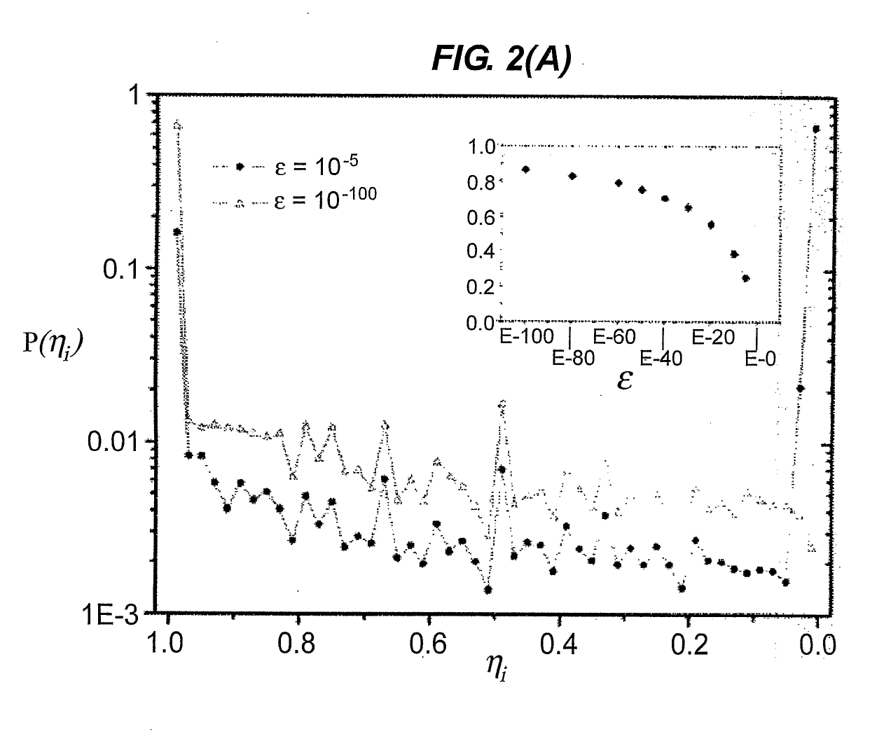

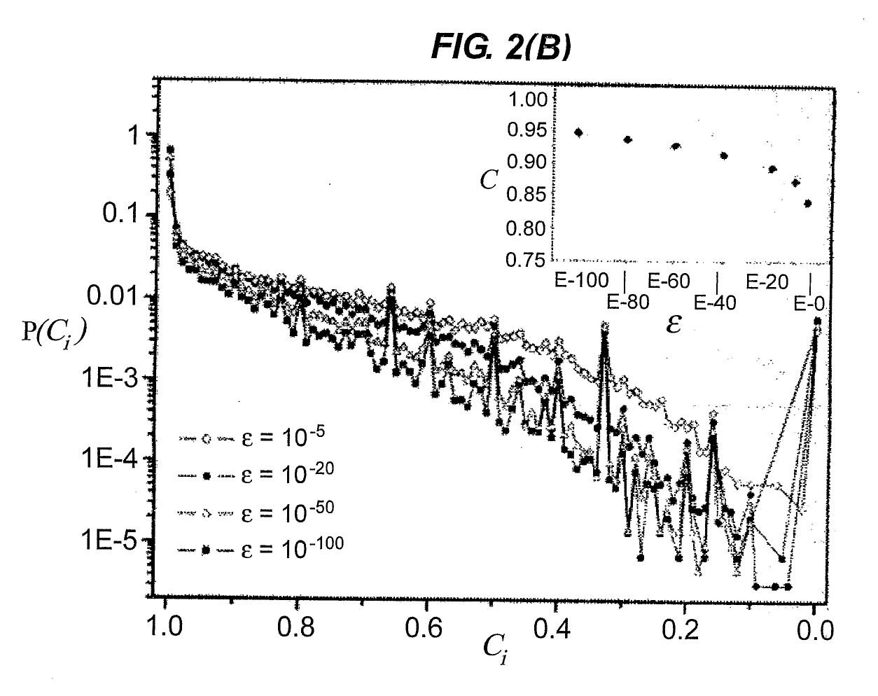

Example 2 demonstrates the behavior of Cᵢ and C for different values of ε using actual protein sequences. The Cᵢ distribution is only slightly dependent upon ε, indicating that the local topology of sequence similarity networks does not depend critically upon the evolutionary distance considered in protein homology relationships. Example 2 further demonstrates that sequence similarity networks are composed of highly connected regions. As shown in FIG. 2A, however, there is a non-negligible fraction of sequences with small clustering indices, indicating that sequence similarity networks include non-compact and even star-like topologies within networks.

Compactness is another useful measure for evaluating the topology of a network and therefore its quality. Compactness can be evaluated using ηᵢ, which is defined as:

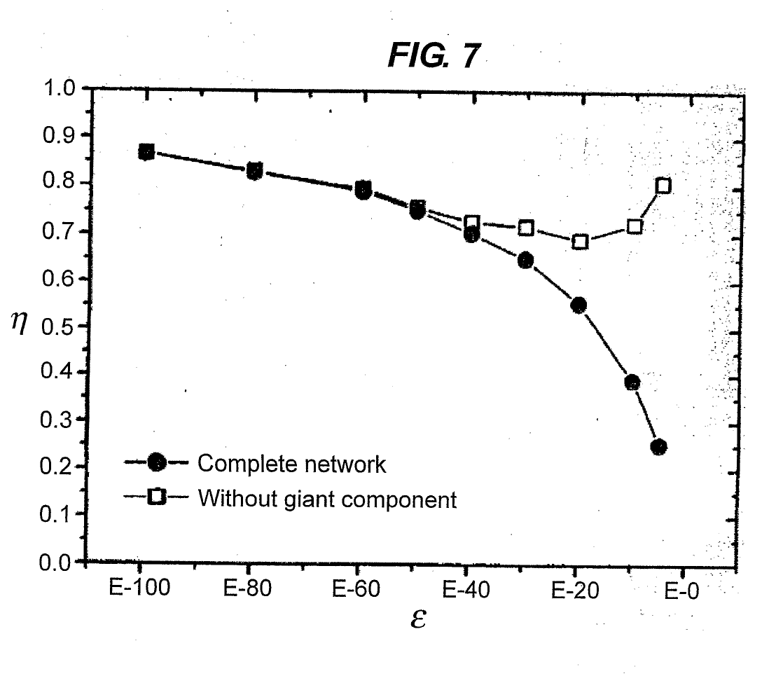

ηᵢ=(kᵢ)/(Mᵢ−1)

where kᵢ is the number of links present in the i-th component and Mᵢ is the number of nodes in the same partition. ηᵢ represents the fraction of nodes in the same partition as the node i that are also the nearest neighbors of i. η is the average over all the nodes ηᵢ: η=(1/N) ηᵢ, where N is number of nodes in the network. Isolated nodes can be excluded from the average. For low values of ε, the sequence similarity networks are composed of compact clusters including only very closely related protein or nucleic acid sequences. With increasing ε, the sequence similarity networks become sparser as more distant homology relations are included. In certain embodiments, a single giant component eventually dominates the network and the compactness index drops sharply. The emergence of a single giant component has been noted in network science and the similarities to critical phenomenon in statistical physics have been studied (22). By excluding the giant component from the average, the behavior of η can change. Instead of the sharp drop in the compactness index, η can initially decrease with increasing ε, but can increase again as connected components not in the giant component become more progressively compact (see FIG. 7 computed using a limited set of the data used in the Examples).

The giant component for all values of ε is characterized by a high degree of compactness, so it is composed of a set of compact regions that are loosely connected by few links. The giant component normally contains more than one biologically meaningful family. A possible cause is the existence of proteins containing more than one functional domain (23, 24, 25). Thus, using sequences that include only a single protein domain can limit the growth of the giant component. Similarly, nucleic acids containing multiple repeated elements will tend to increase the growth of the giant component. Another contributing factor will be links due to sequence similarities that are not of biological origin, i.e. false positives (26).

One of skill in the art can use these measures as well as other measures of network quality available in cluster analysis, to guide the selection of appropriate sequence similarity thresholds in the simplest implementations of the sequence similarity criterion. In addition, one of skill in the art will rely on other factors in selection of the appropriate cut-off. Bioinformaticians are adept in selecting appropriate cutoffs for homology searches given their familiarity with the methods of generating most of the sequence similarity indices. By way of example, BLAST has been used for more than a decade to aid in construction of phylogenetic trees. Thus, selection of percent identity or E-value as a cut-off will be determined, in part, by the nature of the question being asked by the bioinformatician. For example, where only closely related families are of interest, a more restrictive cutoff will be selected whereas a less restrictive cutoff will be used where more distantly related families are of interest. In certain uses of the present methods, a series of increasingly restrictive cutoffs may be used to determine phylogenetic relationships between sequence similarity families. Use of multiple cutoffs can reveal how large families with distantly related sequences are divided into smaller and smaller families as the sequences diverged during evolution.

These measures are also useful for evaluation of new sequence similarity indices or similarity criterion that one of skill in the art may have less familiarity with. By way of example, one of skill in the art could compare the change in the compactness of a sequence similarity generated with different cutoffs of E-value and compare to cutoffs in the less familiar sequence similarity index to apply the appropriate similarity criterion. In addition different sequence similarity criterion may be compared using the above measures to determine which similarity criterion produces the desired results. Where the sequence similarity criterion is a cutoff based upon E-values, the preferred sequence similarity thresholds are about 1, about 10⁻¹, about 10⁻², about 10⁻³, about 10⁻⁴, about 10⁻⁵, about 10⁻⁶, about 10⁻⁷, about 10⁻⁸, about 10⁻¹⁰, about 10⁻¹⁵, about 10⁻²⁰, about 10⁻³⁰, or in the range of about 10⁻¹ to about 10⁻⁴⁰, about 10⁻⁵ to about 10⁻³⁰. Where the sequence similarity criterion is a cutoff based upon percent identity, the preferred sequence similarity thresholds are about 35%, about 40%, about 45%, about 50%, about 60%, about 70%, about 75%, about 80%, about 85%, about 90%, about 95%, or in the range of about 35% to about 95%, or about 45% to about 85% identity.

More complicated sequence similarity criteria may be used in some embodiments to generate the sequence similarity network. Cluster analysis provides numerous examples that may be adapted to the present invention, given the expected distribution of sequences in sequence similarity networks based upon, e.g., evolutionary and functional constraints upon sequence diversity. By way of example, the sequence similarity criterion can involve multiple passes that optimize the network prior to application of the overlap procedure. An example of a reiterative procedure would be to use PSI-BLAST with a reasonable number of reiterative passes (e.g., js=10) where the first iteration E-value is used if convergence is not reached. In addition to merely using primary sequence homology in calculating ε and applying the sequence similarity criteria, predicted secondary structure may be used in mixed or multi-pass homology inference. Non-heuristic sequence similarity searches may also be used such as the Smith-Waterman algorithm.

Overlap Procedure

After a sequence similarity network has been generated, in preferred embodiments, the network is optimized by rewiring to preferentially remove links likely to be incorrect and add links likely to have been missed. In more preferred embodiments, the original sequence similarity network may be retained and the overlap procedure may be applied to partition the sequence similarity network into sequence similarity families which may be in a separate network. Since proteins and nucleic acids within the same family, and therefore within a cluster, should share a large fraction of their nearest neighbors, a preferred method of optimizing uses an overlap criterion that optimizes the sequence similarity network or partitions it into sequence similarity families. In preferred embodiments, the overlap procedure can be used to remove links between nodes that fail to meet an overlap criterion and can also be used to add links between nodes that meet an overlap criterion. For each pair of nodes i, j, the overlap θᵢⱼ may be calculated as:

θᵢⱼ=nᵢⱼ/max(kᵢ,kⱼ)

where nᵢⱼ is the number of nearest neighbors common to node i and node j, and kᵢ and kⱼ are the number of nearest neighbors of node i and node j, respectively. The overlap measure is symmetric, i.e. θᵢⱼ=θⱼᵢ. If two nodes belong to a clique (i.e., a cluster where each node is nearest neighbors with all the other nodes in the cluster), their overlap is 1, while nodes belonging to different communities have small values of overlap. An alternative measure of θᵢⱼ is nᵢⱼ/min(kᵢ, kⱼ) such as was used to analyze the modular structure of metabolic networks (27). However this is less preferred as the former definition is more suited to find nodes connected to all members of distinct communities such as multidomain proteins. Another example of an overlap criterion would be to use a weighted overlap function so that closely shared neighbors will be close to 1 and more distant neighbors will count as less (thereby taking into account the εᵢⱼ value; θᵢⱼ=(Σmin(pᵢₓ, pⱼₓ))/max(kᵢ, kⱼ), where pᵢₓ, and pⱼₓ are the percent identity(/100) between node i and shared neighbor x and between node j and shared neighbor x, respectively.

In certain preferred embodiments, higher cutoffs of ε are used such as ε=10⁻⁵ or ε=1 (for E-value similarity indices), e.g., to include a higher number of homology relations in the sequence similarity network being optimized, though a more restrictive cutoff may be used in other embodiments where more closely related families are of interest. A preferred overlap criterion is to rewire the sequence similarity network by only linking a pair of nodes i, j if and only if θᵢⱼ is greater than a selected threshold of θ.

Where smaller values of θ cut-offs are used, the network may still be dominated by a giant component. By increasing the θ cut-off, the size of the largest cluster can decrease, indicating that the giant component is being disconnected into sets of smaller, very compact sub-networks. After rewiring, η preferably will have increased indicating that quality of the network has improved and with increasing values of θ cut-off, η will tend towards 1. Imposing higher θ cut-offs can be used to identify the core of biological families to identify only those sequences that are most closely related. Lower θ cut-offs may be applied to identify larger, more distantly related families. In preferred embodiments, the overlap threshold will be between about 0.2 and about 0.9, between about 0.3 and about 0.8, between about 0.4 and about 0.6, or will be about 0.5.

Other overlap criteria may also be used. Cluster analysis can provide such alternative overlap criteria. For example, different equations that calculate nearest neighbor overlap may be used, such as equations that provide greater weight for shared neighbors that are more similar to a pair of sequences than shared neighbors that are less similar. In addition, different thresholds may be used for adding and for removing links where simple thresholds are used.

To determine the quality of the clustering procedure and in preferred embodiments, optimize the overlap criterion and overlap threshold used in rewiring, other measures of quality, may be used, e.g., the modularity measure. A preferred equation for calculating modularity Q is (19):

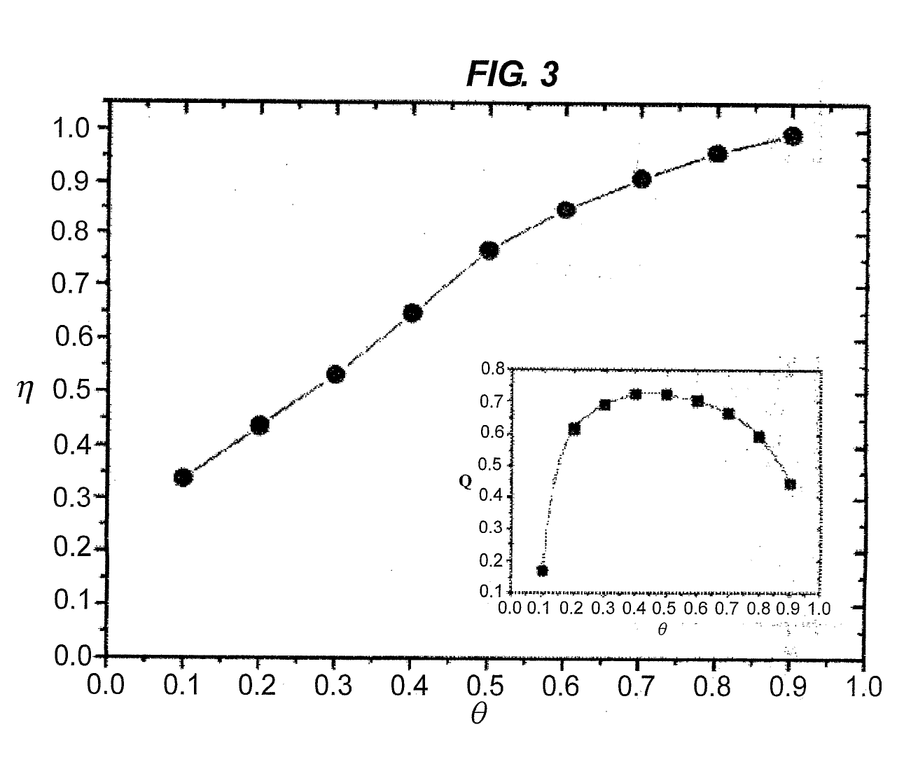

Q=Σ(bᵢ−aᵢ²)

where aᵢ is the fraction of edges with at least one end in the i-th component, and bᵢ is the fraction of edges with both ends in the i-th component. In a random partition of the network, Q=0; values approaching the maximum Q=1 indicate that most of the links are within the components, and therefore the re-wiring or partition of the network captures its underlying modular structure (i.e., the communities are separated). The best values of the modularity index observed in real systems fall in the range of Q=0.3 to 0.7 (19). FIG. 3 (Inset) shows Q at various values of θ cut-off. The curve shows a maximum at around θ=0.5; however, the curve is relatively flat over a fairly wide range of θ cut-offs showing that the several θ cut-offs may be used depending upon whether the desire is to reveal small families of more closely related sequences or larger families that include more distantly related sequences. In preferred embodiments, the overlap cut-off will be yields a modularity coefficient of at least about 0.3, at least about 0.4, at least about 0.5, at least about 0.6, at least about 0.65, or at least about 0.7. In some embodiments overlap threshold selected will yield the highest modularity coefficient.

Identification of Clusters within the Network

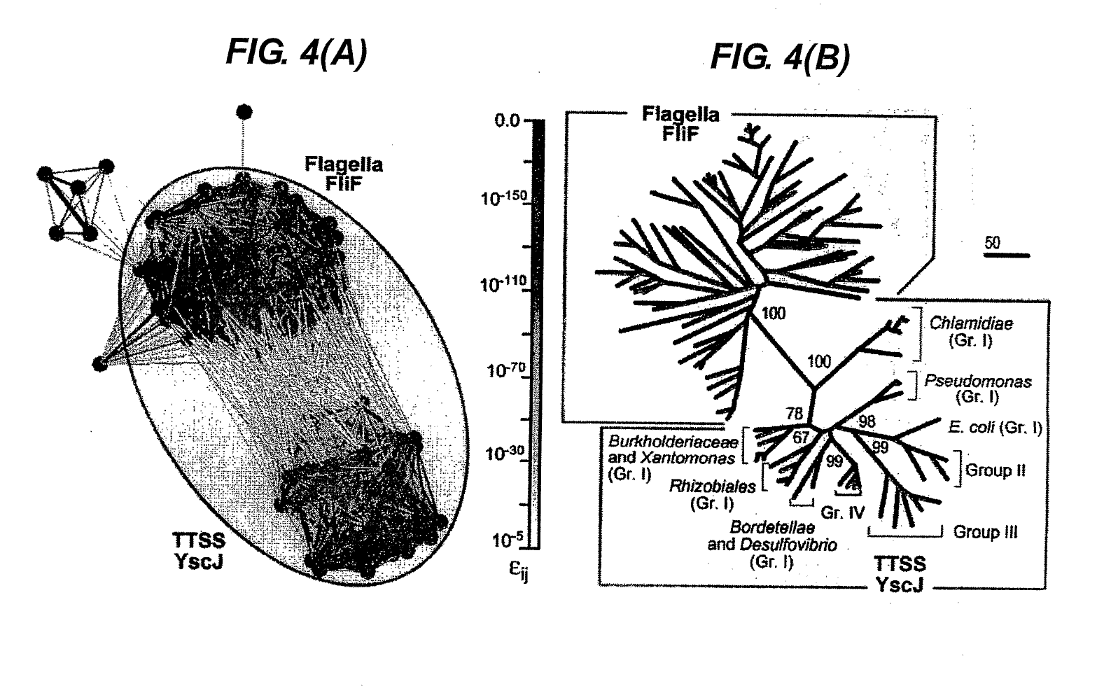

Once the sequence similarity network has been generated, rewiring or partitioning by the overlap procedure preferably removes false links within the network and sequence similarity families become readily identifiable as individual clusters of nodes connected to one another but not to other clusters. Where larger families that include more distantly-related sequences are desired, a lower overlap threshold may be used in the re-wiring procedure. In addition, a more inclusive sequence similarity index cut-off may be used; however, the more inclusive cut-off is the less preferred of the two methods of generating larger families. Similarly, less inclusive cutoffs may be used where small more closely related families are desired. For example, FIG. 4A from the Examples shows two distinct sub-clusters within the larger cluster corresponding to the SctJ sequence similarity family. By using less inclusive cut-offs, these two families may be readily separated. One of skill in the are is well aware of how to select and optimize cut-offs used in identifying sequence similarity families given the similarity in setting cut-offs for traditional phylogenetic tree based organization of related sequences.

Applications

The present invention has a wide range of applications. Being able to group related nucleic acid and protein sequences into families that are related through evolution and/or common function provides a powerful tool to bioinformaticians. The following are preferred examples of applications for the present invention.

Annotation of Known and Novel Sequences

The proliferation of genome sequences from a broad range of organisms has created the daunting task of determining their likely functions. Standard methods of sequence alignment have been used to identify the closest homologs to new sequences to infer likely functional roles; however, such methods typically leave sequences without annotation and may incorrectly annotate a sequence as related to a family when it is not. The robustness of the methods of the present invention can allow more accurate annotation, especially when re-wiring based upon overlap removes false links.

As demonstrated by the Examples below, the methods of the present invention can be applied to multiple genomes simultaneously and can identify members of a family that were not annotated as belonging to the family using traditional sequence alignment methods. With more accurate annotation, one of skill in the art can more readily identify features of a novel sequence such as likely function of a sequence, localization within a cell (e.g., nuclear, cytosolic, membrane bound, etc.), enzymatic activity, if any, (e.g., kinase, tyrosine kinase, phosphatase, metabolic enzyme, etc.), role in a cell (e.g., participates in electron transport, a metabolic pathway, a signaling cascade, etc.), etc. In addition, with more accurate annotations, motifs within a sequence can be more readily identified and validated. For example, a likely role in electron transport would validate identification of mitochondrial targeting sequences, kinase activity would validate identification of nucleotide binding motifs, etc. Sequences with no known role or function may be annotated as well as sequences that have been misannotated.

Identification of Related Protein and Nucleic Acid Sequences

The methods of the present invention are also useful for identifying protein and nucleic acid sequences that are related to a protein or nucleic acid sequence of interest by identifying the sequence similarity family that includes the protein or nucleic acid sequence of interest. By way of example, one may identify proteins that are related to an antigenic protein from a pathogenic virus or bacteria that has been demonstrated to have utility as a component of a vaccine. The related proteins having the same function may also share a similar expression patterns and localization (e.g., exposed on the outer surface of the virus or bacteria and therefore accessible by the host's immune system). Thus, the present methods are useful for identifying novel vaccine targets.

To apply the method, the database of sequences should include the sequence of interest as well as sequences from the target organism. Examples of pathogenic organisms that may provide antigenic proteins of interest or be searched for related proteins include H. pylori, V. cholerae, E. coli, S. typhi, N. gonorrhoeae, N. meningitidis (including individual strains such as A, B, C, Y and W), S. agalactiae (included individual Lancefield classifications designated A to O and individual serotype of each classification), C. pneumoniae, C. trachomatis, HIV (all isolates), rabies viruses, mumps, measles, rubella, polio viruses, FSMB viruses, influenza viruses, Campylobacter, A. trypanosomia, Varicella (Chickenpox), Cryptosporidia, Cyclospora, Arbovirus, West Nile virus, Giardia, Hantavirus, Hepatitis A Virus, Hepatitis B Virus, Hepatitis C Virus, Hepatitis E Virus, Leishmania, H. influenzae, Norovirus, Polio virus, Rickettsia, Rickettsia, Rocky Mountain spotted fever, Rotaviri, S. enteritidis, Coronavirus, Schistosomiasis, Shigella, Streptococcus pneumoniae, Tuberculosis, S. typhi, V. parahaemolyticus, Viral Hemorrhagic Fevers (e.g., Ebola, Lassa, Marburg, Rift Valley), and West Nile virus. In addition to sequences from pathogenic bacteria or viri, sequences from related non-pathogenic strains may be included to improve the accuracy of identification of the sequence similarity family. Once identified, the related sequences in the sequence similarity family may be validated as vaccine components by any number of techniques available to one of skill in the art.

In addition to antigenic proteins, proteins that are likely therapeutic targets or diagnostic molecules may be identified. For example, given that sequence similarity families have the same or similar function, the expression patterns may also be similar and therefore sequences related to a sequence with a diagnostically significant expression pattern will also be likely to have diagnostic significance. In addition surface expressed proteins may also be useful as antibody therapeutic targets and have therefore been the focus of intense research in the field of biotechnology. The present invention can identify surface expressed proteins that would be such likely targets including, e.g., identifying human homologs of targets characterized in other organisms.

Computer Related Embodiments

The various aspects and embodiments of the present invention are particularly amenable to implementation in computer applications and therefore the present invention includes all such aspects and embodiments in the form of computerized systems and computer-readable media that has computer-executable instructions for performing any of the methods of the present invention including without limitation generating or partition a sequence similarity network that has one or more sequence similarity families from a dataset of sequences and annotating sequences within a dataset of sequences. Another preferred aspect includes computerized systems for performing any of the methods of the present invention including without limitation generating or partitioning a sequence similarity network that has one or more sequence similarity families from a dataset of sequences and annotating sequences within a dataset of sequences. Yet another aspect includes computerized systems comprising a computer-readable medium containing a sequence similarity network comprising one or more sequence similarity families generated, partitioned and/or annotated using any of the methods of the present invention.

### Preferred Embodiments

The following examples demonstrate the application of a preferred embodiment of the present invention to two bacterial organelle systems, namely Type III and Type IV Secretion Systems (TTSSs and TFSSs), for which a considerable amount of experimental data is available. TTSSs and TFSSs are contact-dependent export systems widely spread among pathogenic and non-pathogenic bacteria. TTSSs are used by Gram-negative animal and plant pathogens to deliver a wide variety of effector proteins into eukaryotic cells (7). The inner membrane proteins of TTSS share a significant level of homology to components of the assembly machinery of flagella in bacteria, and it has been suggested that the TTSSs have evolved from the more ancient flagellar apparata (8, 9, 10, and 11). TFSSs are transenvelope apparata used by Gram-negative bacteria to translocate proteins and nucleoprotein complexes to recipient cells (12). Some of the energetic and channel components of the TFSS, e.g., the mating-pore formation complex, are highly related to proteins of the Tra/Trb bacterial conjugation systems (13) encoded by several broad-host-range plasmids.

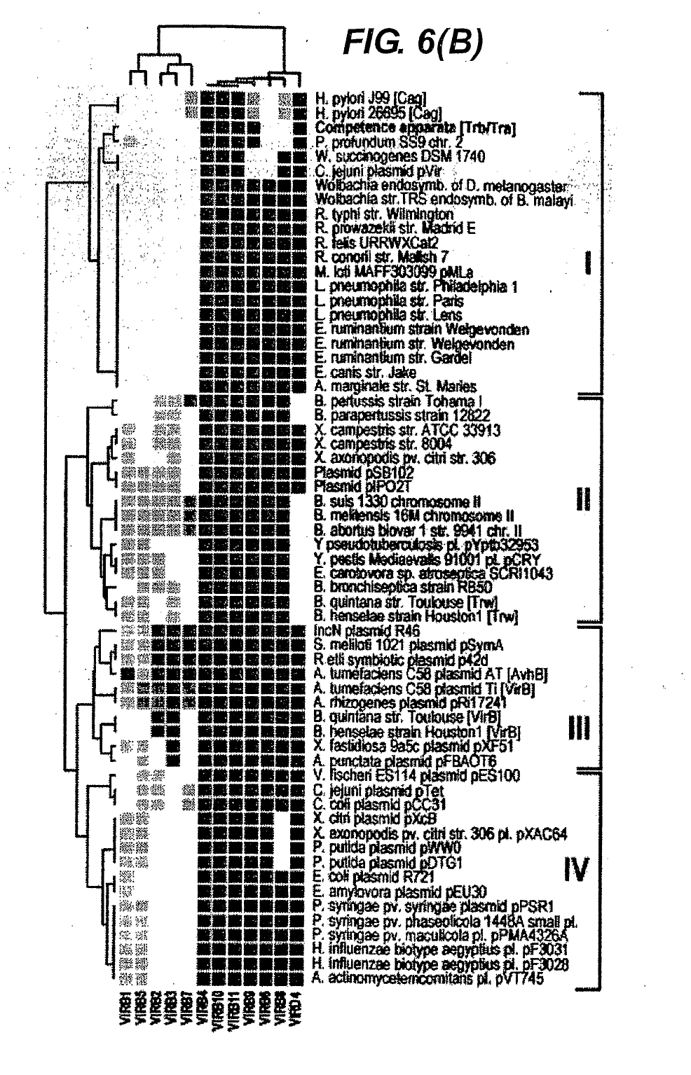

Experimental evidence and comparative analysis of the known apparata including both TTSS (9) and TFSS (14) have been used to define a set of characteristic functions that are conserved in the majority of known apparata and these characteristic functions have been assigned to proteins or sets of proteins that make up the apparata. In some cases, proteins proposed to perform the same function in different apparata show a clear similarity at the level of primary sequence, while in other cases functional homology is inferred by more indirect means such as by similar protein length or conserved genomic context. In the following examples, the proteins of the apparata are partitioned into their respective sequence similarity families. The distribution of the representatives of these functional classes in different sequence similarity families as demonstrated in these examples supports the assignment of the functions to the various proteins and provides an evolutionary based classification of the secretory apparata. This evolutionary based classification highlights the specialization of parts of these organelles to different environments in that the core proteins are conserved across all the apparata while specialized members such as the flagella have additional components that are not found in the core (See FIG. 6).

### Example 1

### Providing the Dataset of Similarity Indices

The amino acid sequences of 761,260 proteins of 256 completely sequenced bacterial genomes and 749 bacterial plasmids were downloaded from the NCBI web site (the complete list is provided in Table 1 below). An all-against-all Blast (21) search was performed, and a matrix containing the Blast E-values was generated. Since the E-value is not invariant for the exchange of the query and target sequences, we defined the symmetric E-value εᵢⱼ between the proteins i, j as εᵢⱼ=min (E-value(i, j), E-value(j, i)).

### Example 2

### Generating the Sequence Similarity Network

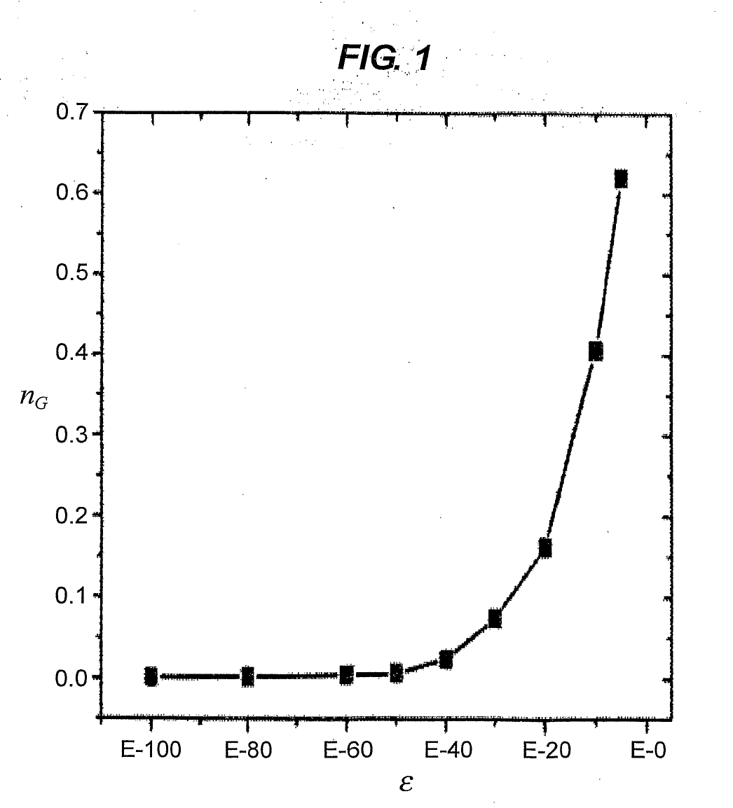

To generate the sequence similarity network, a variety of different cutoffs for εᵢⱼ were tested to maximize the number of links between similar sequences while limiting the number of false similarity links. This effect in the sequence similarity network depends on the value of the homology cut-off ε adopted. For ε=10⁻¹⁸⁰, 1.0·10⁶ links are present. By partitioning the sequence similarity network with a single linkage clustering algorithm, 6.4·10⁵ connected components were found, and 84% of the nodes of the network were singlets, i.e. isolated nodes. With increasing values of ε, more links were included in the network, causing the connected components to merge (See FIG. 1). For ε=10⁻⁵, the highest value of ε considered in this particular example, 6.6·10⁷ links and 8.9·10⁴ connected components were found; singlets included only 8% of the nodes, while the largest connected component contained more than 60% of the whole sequence similarity network. As discussed above, this effect is known as the emergence of the giant component, and the similarities to critical phenomena in statistical physics have been studied (15).

The global structure of the network also changed with ε. In FIG. 2A the distribution of the compactness index ηᵢ is shown for two values of ε. As discussed above, ηᵢ measures the fraction of nodes in its connected component (i.e., in the same partition) to which node i is directly connected (i.e., are nearest neighbors of i). In a clique, all nodes are nearest neighbors, and therefore all have ηᵢ=1, while ηᵢ≈0 if a connected component is sparse. For ε=10⁻¹⁰⁰ more than 70% of the proteins have ηᵢ very close to 1, and therefore the network is dominated by connected components that are very close to cliques. This fraction decreases to less than 20% for ε=10⁻⁵, showing that the network becomes increasingly sparse.

Conversely, the sequence similarity network local structure preserves its biological meaning also for high values of ε, because locally the network still appears as formed by densely interconnected sets of nodes. The local degree of compactness of a network is measured by the clustering index Cᵢ (15), and by its average over the entire network, C. Cᵢ is I for a node at the centre of a fully interlinked region, i.e. if all its nearest neighbors are also directly connected, and tends to 0 for a protein that is part of a loosely connected group. As shown in FIG. 2B, the network in this particular example was always dominated by nodes with high clustering indices. C decreases only from 0.95 for ε=10⁻¹⁸⁰ to 0.84 for ε=10⁻⁵, and also the shape of the distribution of Cᵢ is only slightly dependent on ε, indicating that the sequence similarity network local topology is substantially independent on the evolutionary distance considered in protein homology relations. In a homogeneous random network (16) of the same size and with the same number of links, the clustering index C_{rand} would vary from C_{rand}=1.7·10⁻⁶ to C_{rand}=1.1·10⁻⁴. These results indicate that even at low ε the sequence similarity network is absolutely a non-random network, composed by extremely connected regions, as found in other real world networks (15, 17, 18) where C is comprised in the range 0.1-0.6.

### Example 3

### Optimizing the Network

To optimize the sequence similarity network, the cutoff used in this particular example was ε=10⁻⁵ to maximize the number of links. The sequence similarity network was re-wired by testing different θ cut-offs by connecting two proteins if and only if their overlap θᵢⱼ was smaller than the given cut-off (where 0<θ 1). With this procedure only links connecting nodes that share a certain degree of similarity between their nearest neighbor shells were retained. Nodes belonging to different communities were disconnected, and new links between nodes that were only second nearest neighbors in the original network were introduced.

For small values of θ, the network was still dominated by a single connected component including a large fraction of the nodes (the giant component discussed above). By increasing the cut-off of θ, the size of the largest cluster sharply decreased, and the giant component became disconnected into a set of smaller, compact sub-networks. FIG. 3 shows the compactness index η, re-calculated after the overlap procedure for different values of θ. η grows with θ; for θ=0.5, η=0.77, and for higher values of θ, η tended to the limiting value of η=1, as expected. These values were markedly higher than those obtained before the overlap procedure (see FIG. 2A), and indicate a strict correspondence between the connected components generated by the overlap procedure and the densely interlinked regions of the sequence similarity network.

The extent to which a network re-wiring or partitioning captures the underlying community structure is quantified by the modularity measure Q (19). While for the connected components of the sequence similarity network the maximum was Q=0.39 at ε=10⁻⁴⁰, after the overlap procedure a maximum of Qₘₐₓ=0.723 was obtained, for θ=0.5, as shown in the inset of FIG. 3. Best values of the modularity index observed in real systems fall in the range of Q=0.3 to 0.7 (19), showing that in the sequence similarity network the modular structure was very well defined. Since the value θ=0.5 optimally captured the community structure of the sequence similarity network, the cutoff that two proteins share one half of their nearest neighbors was used in the following examples in order to consider them within the same family or cluster.

For θ=0.5, the network was organized into 34,717 connected components, that were identified as families of similar proteins and constitute sequence similarity-families, plus 127,856 isolated proteins. The giant component of the original homology network was disconnected into 14,443 distinct families plus 26,274 isolated proteins. Eleven percent of the connections were removed from the original homology network, while new links introduced represented about 5% of the connections.

To demonstrate the biological relevance of the overlap procedure, the added and removed links were compared against an external, high quality protein domain classification Pfam (20). It turned out that 98.5% of the newly added links connected proteins that actually share a classified domain according to Pfam, while more than 34% of the removed links involve multi-domain proteins or proteins with non compatible classifications (see Table 1).

Pfam is a curated collection of multiple alignments of protein domains or conserved protein regions. Pfam version 12.0 was used, including 7316 families in Pfam-A and 108,951 in Pfam-B. Proteins are classified in a Pfam family if they own a specific domain. Differently from the sequence similarity families in this example, the same protein can be classified in more than one Pfam family, since a protein can include more than one domain.

A link added to the sequence similarity network by means of the overlap procedure was considered correct if and only if the two connected proteins share at least one Pfam domain. The deletion of a link was considered to be correct if the two connected proteins do not belong to the same Pfam family, or at least one of them is a multi-domain protein.

TABLE 1
Added Links (78.7% testable)

| Protein Classification | Fraction | <θᵢⱼ> |
|---|---|---|
| share a domain | 98.5% | 0.68 |
| do not share a domain | 1.5% | 0.58 |

Removed links (74.7% testable)

| Protein Classification | Fraction | <eᵢⱼ> |
|---|---|---|
| do not share a domain | 8.1% | 10⁻¹⁰ |
| one or two multi-domains | 68.3% | 10⁻⁸⁷ |
| single domain, shared | 23.6% | 10⁻¹⁰ |

The Pfam database includes proteins for 78.7% of the new links introduced and 74.7% of the links removed by the overlap procedure in the sequence similarity network. Of the added links, 98.5% connected proteins sharing at least one domain, confirming the ability of this method to identify distant homologies.

Table 1 also shows the averages of the overlap values for the added links. A lower value was observed for the small fraction of links connecting proteins that did not share an annotated Pfam domain. Of the removed links, 8.1% connected proteins not sharing a PFAM domain, and 68.3% connected at least one multidomain protein. Since the procedure in the example did not classify a protein in more than a family, we consider the deletion of these links as correct. Taken together, these two cases included 76.4% of the removed links. In the remaining 23.6% of the cases, the removed links connected proteins sharing a single domain in Pfam, and therefore the removal of these links are considered incorrect, although the possibility exists that these proteins include domains not yet classified by Pfam.

Also shown in Table 1 are the average E-values of the removed links. Links involving multi-domain proteins are characterized by a much stronger homology than the other removed links.

### Example 4

### Analysis of the Sequence Similarity Families in Contact-Dependent Secretion Systems

The sequence similarity families containing members of the TTSS and TFSS reference functional classes were studied in detail. Table 3 show, for each functional class, the number of the corresponding sequence similarity families and the total number of proteins included in these sequence similarity families. Both TTSS and TFSS are characterized by a core of conserved classes (SctC/J/N/R/S/T/U/V for TTSS, and VirB4/6/8/9/10/11/D4, for TFSS) present in the majority of the systems, each classified in a single sequence similarity family. Core proteins are accompanied by a variable number of accessory proteins belonging to the less conserved functional classes, distributed in multiple sequence similarity families.

TTSSs.

The conserved sequence similarity families in TTSS also contain their flagellar counterparts, indicating that they represent the core machinery common to both systems. The proteins in this group are preferentially localized in the basal body (inner membrane, periplasm and outer membrane), with the exception of SctJ, a lipoprotein whose exact localization is still unclear. After comparing to independent data regarding the functional roles of the proteins, all the proteins classified in the SctV/R/S/T/U/J sequence similarity families belonged either to a TTSS or to a flagellar apparatus. The sizes of these sequence similarity families comprised, between 179 proteins (SctJ) and 229 (SctV). The sequence similarity family including the SctC proteins contained 310 members of the GspD super-family, which in addition to including TTSS and flagellar apparata also include components in competence systems, type II secretion system and type IV pili. The SctN proteins are secretion-specific ATPases included in a large ATP-synthase PHN-family with 973 members. The remaining, less conserved families were much smaller than the conserved ones, going from 25 proteins (SctK, distributed in 2 sequence similarity families), to 181 proteins (SctQ, in 3 sequence similarity families).

As an example, FIG. 4A shows a graphical representation of the region of the sequence similarity containing the SctJ family. Seven proteins with functional annotation incompatible with the SctJ family mediate the connection to the giant component; these outliers were not included in the SctJ family by the overlap procedure. It is worth noting that the links connecting the outliers that were removed by the overlap procedure correspond to a higher level of primary sequence homology than some of the intra-family links within the sequence similarity family that remain after the overlap procedure. For this reason, an analysis of the pair-wise relationships would be hard pressed to recognize the real family structure, thus demonstrating the robustness of the methods of the present invention as compared to the existing methods.

Although all the SctJ proteins, both from TTSSs and flagella, were included in a single sequence similarity family, it is clear from the picture that two sub-structures are present which would likely be separate clusters using more stringent cutoffs. These substructures correspond to the YscJ sub-family of TTSS and to the FliF sub-family of flagellar apparata, respectively. In FIG. 4B a phylogenetic tree of this group of proteins is shown. The same two subgroups identified FIG. 4A form two separate, monophyletic clades of the complete tree, showing that: (i) evolutionary relationships between groups of proteins can be reliably inferred from the topology of the sequence similarity, (ii) sequence similarity families are able to identify distant homology relationships even between compact subgroups.

TFSSs.

Proteins classified in the sequence similarity families were associated with the VirB/D4 reference functional classes belonging either to a TFSS or to a conjugative transfer apparatus. The only exception was the VirB11 proteins which are members of a larger family of ATPases (724 proteins present in a large group of bacteria) used to energize type II and IV secretion systems, type IV pili and competence apparata. The other proteins of the conserved core (VirB4/6/8/9/10/D4) belong, with minor exceptions, each to a single family, containing 69 to 174 proteins. Remaining functional classes showed a lower degree of sequence conservation among different systems, and were split up in 2 (VirB1/5), 3 (VirB3), 4 (VirB2) or 6 (VirB7) different PHN-families. Proteins belonging to the conserved core were known or predicted to be involved in the substrate delivery across one or both membranes, through the so called mating-pore-formation complex (14). Conversely, the majority of the remaining gene products contribute to the formation of the extra-cellular conjugative pilus, or are secreted after post-translational modifications.

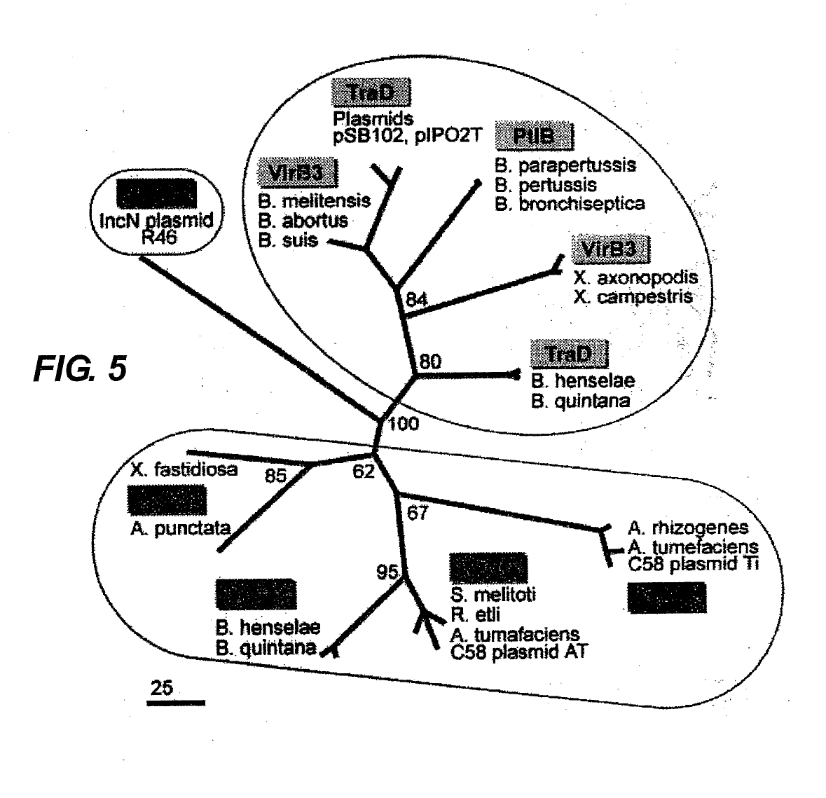

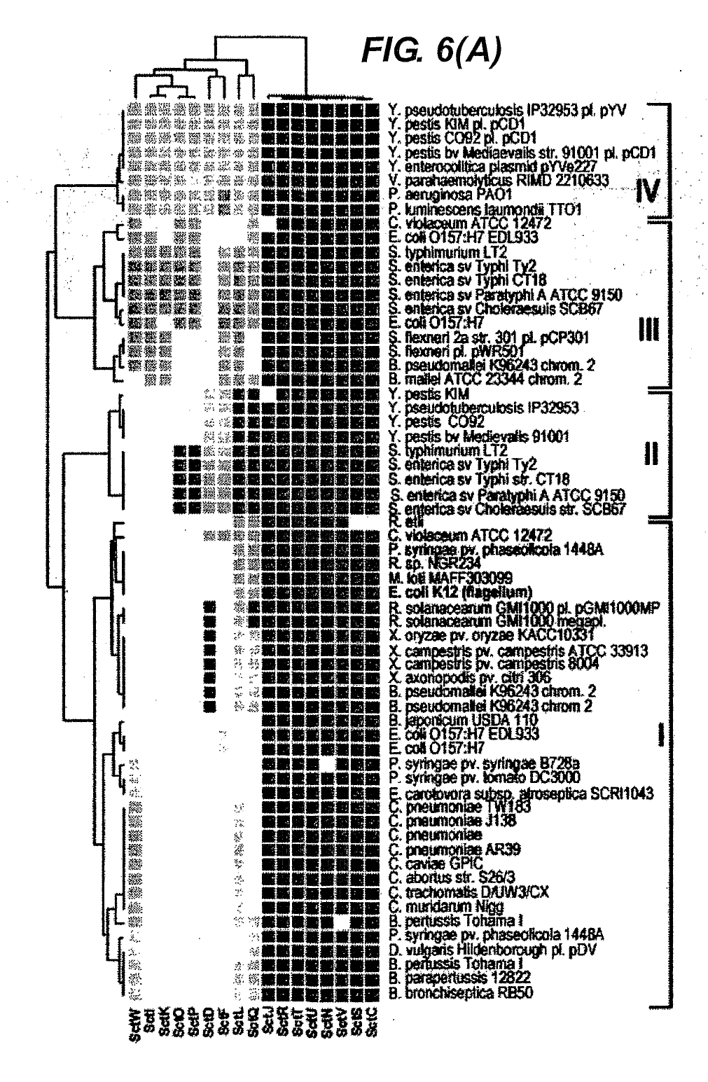

For the 33 VirB3 proteins, a typical example of a non-core family, the phylogenetic tree shown in FIG. 5 shows that each single sequence similarity family corresponds to a monophyletic group. The same is true for the other TT and TFSS families. In the VirB3 case it is interesting to observe that the genetic distance, as measured by molecular phylogenetic analysis, can be higher between members of the same family (X. fastidiosa and Ti plasmid VirB3, 230 point accepted mutations, PAMs) than between members of different families (X. fastidiosa VirB3 and B. henselae TraD, 182 PAMs). This shows that the sequence similarity families capture non trivial evolutionary patterns even when, after the differentiation of two families, family members have undergone sharp, asymmetric genetic divergences.

### Example 5

### Type III and Type IV Secretion Systems Profiling Based on Sequence Similarity Families

The sequence similarity families generated from the reference TT and TFSSs are templates that can be used to identify other secretory apparata. As reference functional classes for TTSS and TFSS, the major structural components of 7 TTSS from 5 bacteria, and 6 TFSS from 4 bacteria and a broad host range plasmid were identified (see Tables 1 and 2 below). TTSS proteins have been classified in seventeen functional groups (SctC/D/F/1-L/N/W) according to the unified nomenclature proposed in (9). TFSS proteins have been classified in twelve functional groups (VirB1-11/D4) using the A. tumefaciens VirB operon as a prototype (12).

TTSSs were identified by requiring that a DNA molecule encode at least one member of five of the conserved families common both to TTSS and to flagella (SctC, SctJ, SctN, SctR, SctS, SctT, SctU, SctV). To distinguish TTSSs from flagellar systems, the molecule was also required to encode also at least one member of one of the families specific to TTSSs (SctD, SctF, SctI, SctK, SctL, SctO, SctP, SctQ).

Similarly, TFSSs were identified by requiring that a DNA molecule encodes at least one member of 5 of the conserved families VirB4/6/8/9/10/11/D4. To distinguish TFSSs from conjugative apparata, the presence of a VirB6 or a non-core protein was required.

By looking for regions that have similar sequence similarity family compositions, 62 putative TTSS in 44 different genomes and 61 putative TFSS in 51 genomes plus 3 broad host range plasmids were identified. A representation of these systems is shown in FIG. 6, where the proteins are color coded according to the sequence similarity family to which they belong. Also shown, is a hierarchical clustering of the different systems based on the sequence similarity family classification of their constituents. The result was a sequence similarity family based profiling of TT and TFSS that allows one of skill in the art to distinguish different groups of secretory apparata.

TTSSs.

Four fundamental groups of TTSS, indicated by the roman numbers I-IV in FIG. 6A, were identified: I) a composite group including the flagellar export machinery in E. coli K12, used as an outgroup; II) the Salmonella SPI-2 system; III) the Salmonella SPI-1 system; and IV) the Yersinia Ysc system of the pCD1 plasmid. Due to the lack of most of the proteins characterizing the TTSSs, group I appears to have evolved early after the speciation of TTSSs from flagellar export apparata. Groups II, III and IV have probably formed later by the recruitment of a variable number of specialized proteins, as confirmed by the molecular phylogenetic analysis on conserved genes (see, for instance, FIG. 4B). Groups II, III, and IV are monophyletic, suggesting that the proteins specific to these groups have been acquired before the speciation of the individual systems. However, it is also evident from FIG. 6A that, while the proteins specific to group IV could have been acquired in a single event, at least two independent horizontal transfer events are required for the formation of systems in group II and III.

TFSSs.

Four groups of TFSSs have been identified as shown in FIG. 6B. Group I includes 33 Tra/Trb identical conjugative apparata (only one representative is shown in the figure) and the H. pylori Cag apparatus, whose VirB7/8/9 genes have differentiated so much from their ancestors that are no longer classified in the respective core families. Group II is characterized by the VirB1/2/3/5 proteins of the pSB102/pIPO2T broad host range plasmids; group III by the VirB3 (and to a minor extent VirB2/7) genes of the A. tumefaciens VirB apparatus; organelles in group IV complement the core set with only one or two accessory proteins (VirB1/5) shared with both the A. tumefaciens VirB and the pSB102/pIPO2T operon. Group IV includes the C. jejuni and C. coli plasmids, whose VirB7 proteins belong to the same small family of the H. pylori Cag (group I). This incongruence, along with the VirB6 small family of the Bordetellae Ptl system and the non-homogeneous pattern of VirB1/2/3/5/7 PHN-families in Agrobacterii, Rhizobii, Bartonellae and Xylellae of group III, again indicates that distinct genetic units have been recruited independently to complement the core proteins.

From the observation of the sequence similarity network topology, it is evident that evolution has induced the living organisms to synthesize only proteins that populate a very small fraction of the protein universe, defined as the set of all the possible sequences that could be obtained by random combinations of the 20 aminoacids. In this “space,” proteins are organized in a fashion that resembles the mass distribution of the physical universe: dense clusters of massive objects separated by sidereal, empty distances. This topological organization is a signature of the evolutionary pressure from the continuous competition in diverse ecological niches. The protein families are the outcome of this selection, marking those regions of the protein space populated by sequences fit to perform biological function conferring a selective advantage to the host organism.

Preferred embodiments of the present invention provide a description of the protein universe, based on the network of sequence similarities, which that allows reconstruction of their evolutionary history and identification of functionally-related proteins.

The coherence of this classification have been assessed by measuring a sharp increase in the quality of network modularity and through comparison with an external, high quality protein domains database (20). The foregoing examples using the sequence similarity family classification have identified and catalogued protein families within the Type III and Type IV secretion systems demonstrating the utility of the present invention.

In both systems, the methods verified the presence of a core of conserved functional classes, preferentially performed by proteins not directly interacting with the host cell, localized in the inner membrane, cytoplasmic and periplasmic space. These proteins are present in all systems, and, even if they belong to evolutionary distant apparata, such as flagellar export systems and TTSSs, they were always classified in a single sequence similarity family. The remaining functional classes, likely involved in host-pathogen interactions, are characterized by a higher degree of heterogeneity. As a consequence, these proteins are classified in smaller, highly coherent sequence similarity families reflecting their functional specialization. The different secretory apparata were compared through the sequence similarity family classification of their components, building a genomic-based taxonomy. The obtained groups correlate with the ecological niche preferentially occupied by the organisms, and are consistent with the molecular phylogeny of the conserved proteins.

Some of the non-core functional classes showed a distribution across the hierarchical groups that are not compatible with the main evolutionary path of the apparata as a whole. This indicates that the secretory apparata have not been acquired in a single event. Rather, a conserved module, unmodified since the original duplication from the flagellar secretory apparata in the case of TTSSs or from the mating pore formation complex of the conjugation machinery in the case of TFSSs, has been complemented during evolution with distinct genetic units, recruited independently to build a variety of specialized contact-dependent secretion systems.

In summary, our analysis of TTSS and TFSS suggests that the methods of the present invention are very efficient in elucidating evolutionary relationships of components of complex structures like secretion machineries, and are therefore useful for generation and detection of patterns of conserved functions amongst bacterial organisms. Given the increasing number of sequenced organisms, such a “landscape view” of the protein universe can also provide useful information in the discovery of novel and previously uncharacterized functions.

The molecular phylogenetic investigations disclosed in these Examples were performed by (i) multiple alignment of proteins included in a given sequence similarity family under investigation (core functional classes) or in sequence similarity families associated with the non-core functional class, in either case using clustalw1.83 (46); (ii) 100 replicate bootstrap resampling of the sequence alignment with SEQBOOT (47); (iii) for each replicate, maximum likelihood phylogeny with PROML (47); (iv) generation of consensus trees with CONSENSE (47), using the majority rule extended; (v) for the original multiple alignment, maximum likelihood phylogeny with PROML (47), (vi) consensus tree topology constraining; and (vii) graphical output with TreeView 1.6.6 (Available on the Internet at taxonomy.zoology.gla.ac.uk under the file rod/rod.html).

It will be evident to those skilled in the art that the invention is not limited to the details of the foregoing illustrative embodiments and that the present invention may be embodied in other specific forms without departing from the spirit or attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

### Example 6

### Use of the Homologs As Vaccine Candidates

The methods disclosed herein may be used to identify likely vaccine candidates by identifying homologs of known antigenic proteins in other pathogenic bacteria. The present methods have been applied to two systems: TTSS and TFSS. Both systems are large protein complexes that reside in the bacterial membrane and therefore have surface exposed antigenic proteins that may be used in vaccines against pathogenic bacteria. To date, a number of proteins in TTSS and TFSS have been identified as potential candidates for vaccine components. By way of example, S. Felek et al. (50) demonstrate that virB9 from Ehrlichia canis is highly immunogenic in dogs and therefore homologs of virB9 are likely vaccine candidates in other pathogenic bacteria. Further, TTSS and TFSS are involved in pathogenicity and therefore can serve as useful diagnostic markers to identify pathogenic strains while not generating false positives from closely related non-pathogenic strains. Finally, the TTSS from Salmonella typhimurium has been used to deliver NY-ESO-1 fused to SopE as a therapeutic cancer vaccine (51). Prior exposure to Salmonella typhimurium may limit the efficacy of this bacteria as means of delivering therapeutic vaccines due to the subject's rapid immune response to the bacteria. Thus, the newly identified homologous TTSS from more rare pathogenic bacteria may be superior candidates to deliver heterologous antigens as vaccines.

Polypeptides.

Representative homologous polypeptides of the TFSS and TTSS are disclosed herein in the sequence listing provided herewith and given the SEQ ID NOs between 1 and 1284. There are thus 1284 amino acid sequences. Certain of polypeptides disclosed in the sequence listing have not previously been identified as components of TFSS or TTSS, respectively. The polypeptides are more fully disclosed on Tables 5 and 7 for TFSS and Tables 6 and 8 for TTSS

The disclosure herein also includes polypeptides comprising amino acid sequences that have sequence identity to the TFSS and TTSS amino acid sequences disclosed in the sequence listing. Depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more). These polypeptides include homologs, orthologs, allelic variants and functional mutants. Typically, 50% identity or more between two polypeptide sequences is considered to be an indication of functional equivalence.

Identity between polypeptides is preferably determined by the Smith-Waterman homology search algorithm as implemented in the MPSRCH program (Oxford Molecular), using an affine gap search with parameters gap open penalty=12 and gap extension penalty=1.

These polypeptides may, compared to the TFSS and TTSS sequences in the sequence listing, include one or more (e.g. 1, 2, 3, 4, 5, 6, 7, 8, 9, 10, etc.) conservative amino acid replacements, i.e., replacements of one amino acid with another which has a related side chain. Genetically-encoded amino acids are generally divided into four families: (1) acidic, i.e., aspartate, glutamate; (2) basic, i.e., lysine, arginine, histidine; (3) non-polar, i.e., alanine, valine, leucine, isoleucine, proline, phenylalanine, methionine, tryptophan; and (4) uncharged polar, i.e., glycine, asparagine, glutamine, cysteine, serine, threonine, and tyrosine.

Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to the TFSS and TTSS sequences of the sequence listing. The polypeptides may also include one or more (e.g. 1, 2, 3, 4, 5, 6, 7, 8, 9, 10, etc.) insertions (e.g. each of 1, 2, 3, 4 or 5 amino acids) relative to the TFSS and TTSS sequences of the sequence listing. Some of these deletions, insertions or substitutions may convert one sequence of the invention to another sequence of the invention. Preferrably such polypeptides will be capable of inducing an immune response against the polypeptide from which they are derived, which may be indicated by antibodies against the polypeptide from which they are derived binding to such polypeptides.

Preferred polypeptides of disclosed are those that are homologous to known antigenic proteins or are polypeptides that are lipidated, that are located in the outer membrane, that are located in the inner membrane, or that are located in the periplasm. Particularly preferred polypeptides are those that fall into more than one of these categories, e.g., lipidated polypeptides that are located in the outer membrane. Lipoproteins may have an N-terminal cysteine to which lipid is covalently attached, following post-translational processing of the signal peptide.

This disclosure also includes fragments of the TFSS and TTSS sequences disclosed in the sequence listing. The fragments should comprise at least n consecutive amino acids from the sequences and, depending on the particular sequence, n is 7 or more (e.g. 8, 10, 12, 14, 16, 18, 20, 22, 24, 26, 28, 30, 35, 40, 45, 50, 60, 70, 80, 90, 100 or more).

The fragment may comprise at least one T-cell or, preferably, a B-cell epitope of the sequence. T- and B-cell epitopes can be identified empirically (e.g., using PEPSCAN; or similar methods), or they can be predicted (e.g., using the Jameson-Wolf antigenic, matrix-based approaches, TEPITOPE, neural networks, OptiMer & EpiMer, ADEPT, Tsites, hydrophilicity, antigenic index, etc.). Other preferred fragments are (a) the N-terminal signal peptides of the TFSS and TTSS sequences disclosed in the sequence listing, (b) the TFSS and TTSS polypeptides, but without their N-terminal signal peptides, (c) the TFSS and TTSS polypeptides, but without their N-terminal amino acid residue.

Further preferred fragments are those common to at least two (e.g. 2, 3, 4 or 5) homologous coding sequences, and in particular those common to homologous coding sequences within the sequence listing.

Other preferred fragments are those that begin with an amino acid encoded by a potential start codon (ATG, GTG, TTG). Fragments starting at the methionine encoded by a start codon downstream of the indicated start codon are polypeptides of the invention.

Polypeptides disclosed herein can be prepared in many ways, e.g., by chemical synthesis (in whole or in part), by digesting longer polypeptides using proteases, by translation from RNA, by purification from cell culture (e.g., from recombinant expression), from the organism itself (e.g., after bacterial culture, or directly from patients), etc. A preferred method for production of peptides <40 amino acids long involves in vitro chemical synthesis. Solid-phase peptide synthesis is particularly preferred, such as methods based on tBoc or Fmoc chemistry. Enzymatic synthesis may also be used in part or in full. As an alternative to chemical synthesis, biological synthesis may be used, e.g., the polypeptides may be produced by translation. This may be carried out in vitro or in vivo.

Biological methods are in general restricted to the production of polypeptides based on L-amino acids, but manipulation of translation machinery (e.g., of aminoacyl tRNA molecules) can be used to allow the introduction of D-amino acids (or of other non-natural amino acids, such as iodotyrosine or methylphenylalanine, azidohomoalamne, etc.). Where D-amino acids are included, however, it is preferred to use chemical synthesis. Polypeptides of the invention may have covalent modifications at the C-terminus and/or N-terminus.

Polypeptides disclosed herein can take various forms (e.g., native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides disclosed herein are preferably provided in purified or substantially purified form, i.e., substantially free from other polypeptides (e.g., free from naturally-occurring polypeptides, but may include one or more other purified polypeptides such as in a multicomponent vaccine composition), particularly from other host cell polypeptides, and are generally at least about 50% pure (by weight), and usually at least about 90% pure, i.e., less than about 50%, and more preferably less than about 10% (e.g. 5%) of a composition is made up of other expressed polypeptides.

Polypeptides disclosed herein are preferably antigenic or immunogenic polypeptides, i.e., polypeptides capable of inducing an immune response against the pathogenic bacteria from which the polypeptide is derived or raising antibodies against the polypeptide from which the antigentic or immunogenic polypeptide is derived.

Polypeptides disclosed herein may be attached to a solid support. Polypeptides of the invention may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

Polypeptides can occur as single chains or associated chains. Polypeptides disclosed herein can be naturally or non-naturally glycosylated (i.e., the polypeptide has a glycosylation pattern that differs from the glycosylation pattern found in the corresponding naturally occurring polypeptide).

Polypeptides disclosed herein may be at least 40 amino acids long (e.g., at least 40, 50, 60, 70, 80, 90, 100, 120, 140, 160, 180, 200, 220, 240, 260, 280, 300, 350, 400, 450, 500 or more). Polypeptides disclosed herein may be shorter than 500 amino acids (e.g., no longer than 40, 50, 60, 70, 80, 90, 100, 120, 140, 160, 180, 200, 220, 240, 260, 280, 300, 350, 400 or 450 amino acids).

This disclosure provides polypeptides comprising a sequence —X—Y— or —Y—X—, wherein: —X— is an amino acid sequence as defined above and —Y— is not a sequence as defined above, i.e., this disclosure provides fusion proteins. Where the N-terminus codon of a polypeptide-coding sequence is not ATG then that codon will be translated as the standard amino acid for that codon rather than as a Met, which occurs when the codon is translated as a start codon.

This disclosure provides a process for producing polypeptides disclosed herein, comprising the step of culturing a host cell under conditions which induce polypeptide expression.

This disclosure provides a process for producing the polypeptides disclosed herein, wherein the polypeptide is synthesized in part or in whole using chemical means.

This disclosure provides a composition comprising two or more polypeptides disclosed herein.

This disclosure also provides a hybrid polypeptide represented by the formula NH₂-A-(—X-L)ₙ-B—COOH, wherein X is a polypeptide disclosed herein, L is an optional linker amino acid sequence, A is an optional N-terminal amino acid sequence, B is an optional C-terminal amino acid sequence, and n is an integer greater than 1. The value n is between 2 and x, and the value of x is typically 3, 4, 5, 6, 7, 8, 9 or 10. Preferably n is 2, 3 or 4; it is more preferably 2 or 3; most preferably, n=2. For each n instances, —X— may be the same or different. For each n instances of (—X-L-), linker amino acid sequence -L- may be present or absent. For instance, when n=2 the hybrid may be NH₂—X₁-L₁-X₂-L₂-COOH, NH₂—X₁-X₂—COOH, NH₂—X₁-L₁-X₂—COOH, NH₂—X₁-X₂-L₂-COOH, etc. Linker amino acid sequence(s)-L- will typically be short (e.g., 20 or fewer amino acids, i.e., 19, 18, 17, 16, 15, 14, 13, 12, 11, 10, 9, 8, 7, 6, 5, 4, 3, 2, 1). Examples include leader sequences to direct polypeptide trafficking, or short peptide sequences which facilitate cloning or purification such as poly-glycine linkers (i.e., Gly where n=2, 3, 4, 5, 6, 7, 8, 9, 10 or more) and histidine tags (i.e., His where n 3, 4, 5, 6, 7, 8, 9, 10 or more). Other suitable linker amino acid sequences will be apparent to those skilled in the art. -A- and —B— are optional sequences which will typically be short (e.g., 40 or fewer amino acids, i.e., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1).

Various tests can be used to assess the in vivo immunogenicity of polypeptides of the invention. For example, polypeptides can be expressed recombinantly and used to screen patient sera by immunoblot. A positive reaction between the polypeptide and patient serum indicates that the patient has previously mounted an immune response to the protein in question, i.e., the protein is an immunogen. Thus, preferred polypeptides disclosed herein are polypeptides from pathogenic bacteria that are recognized by an antibody from the sera of a subject that has been exposed to the pathogenic bacteria or the polypeptide. This method can also be used to identify immunodominant proteins.

Antibodies.

This disclosure provides antibodies that bind to polypeptides of the sequence listing. These may be polyclonal or monoclonal and may be produced by any suitable means (e.g., by recombinant expression). To increase compatibility with the human immune system, the antibodies may be chimeric or humanized, or fully human antibodies may be used. The antibodies may include a detectable label (e.g., for diagnostic assays). Antibodies of the invention may be attached to a solid support. Antibodies of the invention are preferably neutralizing antibodies.

Monoclonal antibodies are particularly useful in identification and purification of the individual polypeptides against which they are directed. Monoclonal antibodies of the invention may also be employee as reagents in immunoassays, radioimmunoassays (RIA) or enzyme-linked immunosorbent assays (ELISA), etc. In these applications, the antibodies can be labeled with an analytically detectable reagent such as a radioisotope, a fluorescent molecule or an enzyme. The monoclonal antibodies produced by the above method may also be used for the molecular identification and characterization (epitope mapping) of polypeptides of the invention.

Antibodies disclosed herein are preferably specific to the strain the polypeptide was derived from, i.e., they bind preferentially to the parent bacteria relative to other bacteria. Antibodies disclosed herein are preferably provided in purified or substantially purified form.

Typically, the antibody will be present in a composition that is substantially free of other polypeptides e.g. where less than 90% (by weight), usually less than 60% and more usually less than 50% of the composition is made up of other polypeptides.

Antibodies disclosed herein can be of any isotype (e.g., IgA, IgG, IgM, etc., i.e., an α, γ, or μ heavy chain), but will generally be IgG. Within the IgG isotype, antibodies may be IgG1, IgG2, IgG3 or IgG4 subclass. Antibodies disclosed herein may have a κ- or λ-light chain.

Antibodies disclosed herein can take various forms, including whole antibodies, antibody fragments such as F(ab′)2 and F(ab) fragments, Fv fragments (non-covalent heterodimers), single-chain antibodies such as single chain Fv molecules (scFv), minibodies, oligobodies, etc. The term “antibody” does not imply any particular origin, and includes antibodies obtained through non-conventional processes, such as phage display.

This disclosure provides a process for detecting polypeptides disclosed herein, comprising the steps of: (a) contacting an antibody disclosed herein with a biological sample under conditions suitable for the formation of an antibody-antigen complexes; and (b) detecting said complexes.

This disclosure provides a process for detecting antibodies disclosed herein, comprising the steps of: (a) contacting a polypeptide disclosed herein with a biological sample (e.g., a blood or serum sample) under conditions suitable for the formation of an antibody-antigen complexes; and (b) detecting said complexes.

For good cross-reactivity, preferred antibodies are common to at least two (e.g., 2, 3, 4 or 5) homologous coding sequences, as described in more detail above. Conversely, for good specificity, other preferred antibodies disclosed herein bind to epitopes that include an amino acid that differs between homologous coding sequences.

Nucleic Acids.

This disclosure provides nucleic acid comprising the nucleotide sequences disclosed in the sequence listing. These nucleic acid sequences are the nucleic acids encoding the polypeptides of SEQ ID NOs between 1 and 1284.

This disclosure also provides nucleic acid comprising nucleotide sequences having sequence identity to the nucleic acids encoding the TFSS and TTSS polypeptides disclosed in the sequence listing or otherwise disclosed herein. Identity between sequences is preferably determined by the Smith-Waterman homology search algorithm as described above.

This disclosure also provides nucleic acid which can hybridize to the GBS nucleic acid disclosed in the examples. Hybridization reactions can be performed under conditions of different “stringency.”

Conditions that increase stringency of a hybridization reaction of widely known and published in the art. Examples of relevant conditions include (in order of increasing stringency): incubation temperatures of 25° C., 37° C., 50° C., 55° C. and 68° C.; buffer concentrations of x SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art.

In some embodiments, nucleic acids disclosed herein hybridizes to a target sequence in the sequence listing under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC. Each of the foregoing wash conditions preferably are performed for twenty minutes.

Nucleic acid comprising fragments of these sequences are also provided. These should comprise at least n consecutive nucleotides from the GBS sequences and, depending on the particular sequence, n is 10 or more (e.g. 12, 14, 15, 18, 20, 25, 30, 35, 40, 50, 60, 70, 80, 90, 100, 150, 200 or more).

This disclosure provides nucleic acid of formula 5′-X-Y-Z-3′, wherein: —X— is a nucleotide sequence consisting of x nucleotides; -Z- is a nucleotide sequence consisting of z nucleotides; —Y— is a nucleotide sequence consisting of either (a) a fragment of one of the nucleic acids encoding SEQ ID NOs: 1 to 1284, or (b) the complement of (a); and said nucleic acid 5′-X-Y-Z-3′ is neither (i) a fragment of one of the nucleic acids encoding SEQ ID NOs: 1 to 1284 nor (ii) the complement of (i). The —X— and/or -Z-moieties may comprise a promoter sequence (or its complement).

This disclosure also provides nucleic acid encoding the polypeptides and polypeptide fragments disclosed herein.

This disclosure includes nucleic acid comprising sequences complementary to the sequences encoding the polypeptides in the sequence listing (e.g., for antisense or probing, or for use as primers), as well as the sequences in the coding orientation.

Nucleic acids of disclosed herein can be used in hybridization reactions (e.g., Northern or Southern blots, or in nucleic acid microarrays or ‘gene chips’) and amplification reactions (e.g., PCR, SDA, SSSR, LCR, TMA, NASBA, etc.) and other nucleic acid techniques.

Nucleic acid disclosed herein can take various forms (e.g., single-stranded, double-stranded, vectors, primers, probes, labeled, etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids disclosed herein are preferably provided in purified or substantially purified form, i.e., substantially free from other nucleic acids (e.g., free from naturally-occurring nucleic acids), particularly from other host cell nucleic acids, generally being at least about 50% pure (by weight), and usually at least about 90% pure. Nucleic acids of the invention are preferably pathogenic bacterial nucleic acids.

Nucleic acids disclosed herein may be prepared in many ways, e.g., by chemical synthesis (e.g., phosphoramidite synthesis of DNA) in whole or in part, by digesting longer nucleic acids using nucleases (e.g., restriction enzymes), by joining shorter nucleic acids or nucleotides (e.g., using ligases or polymerases), from genomic or cDNA libraries, etc. Nucleic acids disclosed herein may be attached to a solid support (e.g., a bead, plate, filter, film, slide, microarray support, resin, etc.). Nucleic acids disclosed herein may be labeled, e.g., with a radioactive or fluorescent label, or a biotin label. This is particularly useful where the nucleic acid is to be used in detection techniques, e.g., where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g., peptide nucleic acids (PNAs) or phosphorothioates) or modified bases. Thus this disclosure includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids disclosed herein comprise the sequences disclosed herein, but they may also comprise other sequences (e.g., in nucleic acids of formula 5′-X-Y-Z-3′, as defined above). This is particularly useful for primers, which may thus comprise a first sequence complementary to a disclosed nucleic acid target and a second sequence which is not complementary to the disclosed nucleic acid target. Any such non-complementary sequences in the primer are preferably 5′ to the complementary sequences. Typical non-complementary sequences comprise restriction sites or promoter sequences.

Nucleic acids disclosed herein may be part of a vector, i.e., part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors,” which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acids disclosed herein.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T). The terms also imply a direction—the complement of 5′-ACAGT-3′ is 5′-ACTGT-3′ rather than 5′-TGTCA-3′.

Nucleic acids disclosed herein can be used, for example: to produce polypeptides; as hybridization probes for the detection of nucleic acid in biological samples; to generate additional copies of the nucleic acids; to generate ribozymes, antisense or siRNA oligonucleotides; as single-stranded DNA primers or probes; or as triple-strand forming oligonucleotides.

This disclosure provides a process for producing nucleic acids disclosed herein, wherein the nucleic acid is synthesized in part or in whole using chemical means.

This disclosure provides vectors comprising nucleotide sequences of the invention (e.g., cloning or expression vectors) and host cells transformed with such vectors.

This disclosure also provides a kit comprising primers (e.g., PCR primers) for amplifying and/or detecting a template sequence contained within a pathogenic bacterium nucleic acid sequence, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label (e.g., a fluorescent label).

This disclosure also provides a kit comprising first and second single-stranded oligonucleotides which allow amplification of a template nucleic acid sequence disclosed herein contained in a single- or double-stranded nucleic acid (or mixture thereof), wherein: (a) the first oligonucleotide comprises a primer sequence which is substantially complementary to said template nucleic acid sequence; (b) the second oligonucleotide comprises a primer sequence which is substantially complementary to the complement of said template nucleic acid sequence; (c) the first oligonucleotide and/or the second oligonucleotide comprise(s) sequence which is not complementary to said template nucleic acid; and (d) said primer sequences define the termini of the template sequence to be amplified. The non-complementary sequence(s) of feature (c) are preferably upstream of (i.e., 5′ to) the primer sequences. One or both of these (c) sequences may comprise a restriction site or a promoter sequence. The first oligonucleotide and/or the second oligonucleotide may include a detectable label (e.g., a fluorescent label).

This disclosure provides a process for detecting nucleic acids disclosed herein, comprising the steps of: (a) contacting a nucleic probe according to the invention with a biological sample under hybridizing conditions to form duplexes; and (b) detecting said duplexes.

This disclosure provides a process for detecting a pathogenic bacteria in a biological sample (e.g., blood), comprising the step of contacting a nucleic acid disclosed herein with the biological sample under hybridizing conditions. The process may involve nucleic acid amplification (e.g., PCR, SDA, SSSR, LCR, TMA, NASBA, etc.) or hybridization (e.g., microarrays, blots, hybridization with a probe in solution etc.). PCR detection of pathogenic bacteria in clinical samples has been reported.

This disclosure provides a process for preparing a fragment of a target sequence, wherein the fragment is prepared by extension of a nucleic acid primer. The target sequence and/or the primer are nucleic acids disclosed herein. The primer extension reaction may involve nucleic acid amplification (e.g., PCR, SDA, SSSR, LCR, TMA, NASBA, etc.).

Nucleic acid amplification as disclosed herein may be quantitative and/or real-time.

For certain embodiments, nucleic acids are preferably at least 7 nucleotides in length (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments, nucleic acids are preferably at most 500 nucleotides in length (e.g., 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g., 10, 11, 12, 13, 14, 15, 16, 17, 18, 19, 20, 21, 22, 23, 24, 25, 26, 27, 28, 29, or 30 nucleotides).

Pharmaceutical Compositions.

This disclosure provides compositions comprising: (a) polypeptide, antibody, and/or nucleic acid of the invention; and (b) a pharmaceutically acceptable carrier. These compositions may be suitable as immunogenic compositions, for instance, or as diagnostic reagents, or as vaccines. Vaccines according to the invention may either be prophylactic (i.e., to prevent infection) or therapeutic (i.e., to treat infection), but will typically be prophylactic.

A “pharmaceutically acceptable carrier” includes any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier.

Compositions disclosed herein may include an antimicrobial, particularly if packaged in a multiple dose format.

Compositions disclosed herein may comprise detergent, e.g., a Tween (polysorbate), such as Tween 80. Detergents are generally present at low levels, e.g., >0.01%.

Compositions disclosed herein may include sodium salts (e.g., sodium chloride) to give tonicity. A concentration of 10±2 mg/ml NaCl is typical.

Compositions disclosed herein will generally include a buffer. A phosphate buffer is typical.

Compositions disclosed herein may comprise a sugar alcohol (e.g., mannitol) or a disaccharide (e.g., sucrose or trehalose), e.g., at around 15-30 mg/ml (e.g., 25 mg/ml), particularly if they are to be lyophilized or if they include material which has been reconstituted from lyophilized material. The pH of a composition for lyophilization may be adjusted to around 6.1 prior to lyophilization.

Polypeptides of disclosed herein may be administered in conjunction with other immunoregulatory agent. In particular, compositions will usually include a vaccine adjuvant. Adjuvants which may be used in compositions disclosed herein include, but are not limited to:

A. Mineral-Containing Compositions

Mineral containing compositions suitable for use as adjuvants in the disclosed compositions include mineral salts, such as aluminum salts and calcium salts. The adjuvants include mineral salts such as hydroxides (e.g., oxyhydroxides), phosphates (e.g., hydroxyphosphates, orthophosphates), sulphates, or mixtures of different mineral compounds (e.g., a mixture of a phosphate and a hydroxide adjuvant, optionally with an excess of the phosphate), with the compounds taking any suitable form (e.g., gel, crystalline, amorphous, etc.), and with adsorption to the salt(s) being preferred. Mineral containing compositions may also be formulated as a particle of metal salt.

Aluminum salts may be included in vaccines disclosed herein such that the dose of Al³⁺ is between 0.2 and 1.0 mg per dose.

A typical aluminum phosphate adjuvant is amorphous aluminum hydroxyphosphate with PO₄/A molar ratio between 0.84 and 0.92, included at 0.6 mg Al³⁺/ml. Adsorption with a low dose of aluminum phosphate may be used, e.g., between 50 and 100 μg Al³⁺ per conjugate per dose. Where an aluminum phosphate is used and it is desired not to adsorb an antigen to the adjuvant, this is favored by including free phosphate ions in solution (e.g., by the use of a phosphate buffer).

B. Oil Emulsions

Oil emulsion compositions suitable for use as adjuvants include squalene-water emulsions, such as MF59 (5% Squalene, 0.5% Tween 80, and 0.5% Span 85, formulated into submicron particles using a microfluidizer). MF59 is used as the adjuvant in the FLUAD™ influenza virus trivalent subunit vaccine.

Particularly preferred adjuvants for use in the compositions are submicron oil-in-water emulsions.

Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80 (polyoxyethylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-sn-glycero-3-hydroxyphosphosphoryloxy)-ethylamine (MTP-PE). Submicron oil-in-water emulsions, methods of making the same and immunostimulating agents, such as muramyl peptides, for use in the compositions, are available in the art.

Complete Freund's adjuvant (CFA) and incomplete Freund's adjuvant (IFA) may also be used as adjuvants in the compositions disclosed herein.

C. Saponin Formulations

Saponin formulations may also be used as adjuvants in the invention. Saponins are a heterologous group of sterol glycosides and triterpenoid glycosides that are found in the bark, leaves, stems, roots even-flowers of a wide range of plant species. Saponins isolated from the of the Quillaja saponaria Molina tree have been widely studied as adjuvants. Saponin can also be commercially obtained from Smilax ornata (sarsaparilla), Gypsophilla paniculata (brides veil), and Saponaria officinalis (soap root). Saponin adjuvant formulations include purified formulations, such as QS21, as well as lipid formulations, such as ISCOMs.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. Saponin formulations may also comprise a sterol, such as cholesterol.

Combinations of saponins and cholesterols can be used to form unique particles called immunostimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA and QHC. Optionally, the ISCOMs may be devoid of additional detergent(s).

D. Virosomes and Virus-Like Particles

Virosomes and virus-like particles (VLPs) can also be used as adjuvants in the compositions disclosed herein. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis B virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1).

E. Bacterial or Microbial Derivatives

Adjuvants suitable for use in the compositions disclosed herein include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. Preferred “small particle” forms of 3 de-O-acylated monophosphoryl lipid A are available in the art. Such “small particles” of 3dMPL are small enough to be sterile filtered through a 0.22 μm membrane. Other non-toxic LPS derivatives include monophosphoryl lipid A mimics, such as aminoalkyl glucosaminide phosphate derivatives, e.g., RC-529.

Lipid A derivatives include derivatives of lipid A from Escherichia coli such as OM-174.

Immunostimulatory oligonucleotides suitable for use as adjuvants with the disclosed compositions include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. Analog substitutions such as replacement of guanosine with 2′-deoxy-7-deazaguanosine may also be used.

The CpG sequence may be directed to TLR9, such as the motif GTCGTT or TTCGTT. The CpG sequence may be specific for inducing a Th1 immune response, such as a CpG-A ODN, or it may be more specific for inducing a B cell response, such a CpU-B ODN. Preferably, the CpG is a CpG-A ODN.

Preferably, the CpG oligonucleotide is constructed so that the 5′ end is accessible for receptor recognition. Optionally, two CpU oligonucleotide sequences may be attached at their 3′ ends to form “immunomers.”

Bacterial ADP-ribosylating toxins and detoxified derivatives thereof may be used as adjuvants in the invention. Preferably, the protein is derived from E. coli (E. coli heat labile enterotoxin “LT”), cholera toxin, or pertussis toxin. The use of detoxified ADP-ribosylating toxins as mucosal adjuvants is has been described in the art and as parenteral adjuvants as well. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in the art.

F. Human Immunomodulators

Human immunomodulators suitable for use as adjuvants in the compositions disclosed herein include cytokines, such as interleukins (e.g., IL-1, IL-2, IL-4, IL-5, IL-6, IL-7, IL-12, etc.), interferons (e.g., interferon-γ), macrophage colony stimulating factor, and tumor necrosis factor.

G. Bioadhesives and Mucoadhesives

Bioadhesives and mucoadhesives may also be used as adjuvants in the compositions disclosed herein. Suitable bioadhesives include esterified hyaluronic acid microspheres; or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the disclosed compositions.

H. Microparticles

Microparticles may also be used as adjuvants in the disclosed compositions. Microparticles (i.e., a particle of 100 nm to ˜450 μm in diameter, more preferably ˜200 nm to ˜300 μm in diameter, and most preferably ˜500 nm to ˜10 μm in diameter) formed from materials that are biodegradable and non-toxic (e.g., a poly(α-hydroxy acid), a polyhydroxybutyric acid, a polyorthoester, a polyanhydride, a polycaprolactone, etc.), with poly(lactide-co-glycoside) are preferred, optionally treated to have a negatively charged surface (e.g., with SDS) or a positively-charged surface (e.g., with a cationic detergent, such as CTAB).

I. Liposomes

Liposome formulations suitable for use as adjuvants may be found throughout the art.

J. Polyoxyethylene Ether and Polyoxyethylene Ester Formulations

Adjuvants suitable for use in the disclosed compositions include polyoxyethylene ethers and polyoxyethylene esters. Such formulations further include polyoxyethylene sorbitan ester surfactants in combination with an octoxynol as well as polyoxyethylene alkyl ethers or ester surfactants in combination with at least one additional non-ionic surfactant such as an octoxynol. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

K. Polyphosphazene (PCPP)

PCPP formulations are available in the art.

L. Muramylpeptides

Examples of muramyl peptides suitable for use as adjuvants in the disclosed compositions include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

M. Imidazoquinolone Compounds

Examples of imidazoquinolone compounds suitable for use adjuvants in the disclosed compounds include Imiquamod and its homologues (e.g., “Resiquimod 3M”).

N. Thiosemicarbazone Compounds

Examples of thiosemicarbazone compounds, as well as methods of formulating, manufacturing, and screening for compounds all suitable for use as adjuvants in the disclosed compositions may be found in the art. The thiosemicarbazones are particularly effective in the stimulation of human peripheral blood mononuclear cells for the production of cytokines, such as TNF-α.

O. Tryptanthrin Compounds

Examples of tryptanthrin compounds, as well as methods of formulating, manufacturing, and screening for compounds all suitable for use as adjuvants in disclosed compositions may be found in the art. The tryptanthrin compounds are particularly effective in the stimulation of human peripheral blood mononuclear cells for the production of cytokines, such as TNF-α.

The disclosed compositions may also comprise combinations of aspects of one or more of the adjuvants identified above. For example, the following combinations may be used as adjuvant compositions in the invention: (1) a saponin and an oil-in-water emulsion; (2) a saponin (e.g., QS21)+a non-toxic LPS derivative (e.g., 3dMPL), a saponin (e.g., QS21)+a non-toxic LPS derivative (e.g., 3dMPL)+a cholesterol; (4) a saponin (e.g., QS21)+3dMPL+IL-12 (optionally+a sterol); (5) combinations of 3dMPL with, for example, QS21 and/or oil-in-water emulsions; (6) SAF, containing 10% squalane, 0.4% Tween 80%, 5% pluronic-block polymer L₁₂₁, and thr-MDP, either microfluidized into a submicron emulsion or vortexed to generate a larger particle size emulsion; (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (8) one or more mineral salts (such as an aluminum salt)+a non-toxic derivative of LPS (such as 3dMPL); and (9) one or more mineral salts (such as an aluminum salt)+an immunostimulatory oligonucleotide (such as a nucleotide sequence including a CpG motif).

The use of an aluminum hydroxide or aluminum phosphate adjuvant is particularly preferred, and antigens are generally adsorbed to these salts. Calcium phosphate is another preferred adjuvant.

The pH of compositions disclosed herein is preferably between 6 and 8, preferably about 7. Stable pH may be maintained by the use of a buffer. Where a composition comprises an aluminum hydroxide salt, it is preferred to use a histidine buffer. The composition may be sterile and/or pyrogen-free. Compositions disclosed herein may be isotonic with respect to humans.

Compositions may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form (e.g., freeze-dried) for solution or suspension in liquid vehicles prior to injection.

Compositions disclosed herein may be packaged in unit dose form or in multiple dose form. For multiple dose forms, vials are preferred to pre-filled syringes. Effective dosage volumes can be routinely established, but a typical human dose of the composition for injection has a volume of 0.5 ml.

Where a composition disclosed herein is to be prepared extemporaneously prior to use (e.g., where a component is presented in lyophilized form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By “immunologically effective amount,” it is meant that the administration of that amount to an individual, either in a single dose or as part of a series, is effective for treatment or prevention. This amount varies depending upon the health and physical condition of the individual to be treated, age, the taxonomic group of individual to be treated (e.g., non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

Pharmaceutical Uses

This disclosure also provides a method of treating a subject, comprising administering to the subject a therapeutically effective amount of a composition disclosed herein. The subject may either be at risk from the disease themselves or may be a pregnant woman (maternal immunization).

This disclosure provides nucleic acid, polypeptide, or antibody disclosed herein for use as medicaments (e.g., as immunogenic compositions or as vaccines) or as diagnostic reagents. It also provides the use of nucleic acid, polypeptide, or antibody disclosed herein in the manufacture of: (i) a medicament for treating or preventing disease and/or infection caused by a pathogenic bacteria; (ii) a diagnostic reagent for detecting the presence of a pathogenic bacteria or of antibodies raised against a pathogenic bacteria; and/or (iii) a reagent which can raise antibodies against a pathogenic bacteria. Said pathogenic bacteria can be of any serotype or strain of pathogenic bacteria disclosed herein.

The subject is preferably a human. Where the vaccine is for prophylactic use, the human is preferably an adolescent (e.g., aged between 10 and 20 years); where the vaccine is for therapeutic use, the human is preferably an adult. A vaccine intended for children or adolescents may also be administered to adults, e.g., to assess safety, dosage, immunogenicity, etc. One way of checking efficacy of therapeutic treatment involves monitoring bacterial infection after administration of the composition of the invention. One way of checking efficacy of prophylactic treatment involves monitoring immune responses against an administered polypeptide after administration. Immunogenicity of compositions of the invention can be determined by administering them to test subjects (e.g., children 12-16 months' age, or animal models, e.g., a mouse model) and then determining standard parameters including ELISA titers (GMT) of IgG. These immune responses will generally be determined around 4 weeks after administration of the composition, and compared to value determined before administration of the composition. Where more than one dose of the composition is administered, more than one post-administration determination may be made.

Administration of polypeptide antigens is a preferred method of treatment for inducing immunity.

Administration of antibodies of the invention is another preferred method of treatment. This method of passive immunization is particularly useful for newborn children or for pregnant women. This method will typically use monoclonal antibodies, which will be humanized or fully human.

Preferred compositions for use in immunization include more than one polypeptide, which can include one polypeptide disclosed with other polypeptides available in the art or more than one polypeptide disclosed herein. Multiple antigens can be included as separate admixed polypeptides in a single composition, and/or can be part of a hybrid polypeptide as described above.

Compositions disclosed herein will generally be administered directly to a subject. Direct delivery may be accomplished by parenteral injection (e.g., subcutaneously, intraperitoneally, intravenously, intramuscularly, or to the interstitial space of a tissue), or by rectal, oral, vaginal, topical, transdermal, intranasal, sublingual, ocular, aural, pulmonary or other mucosal administration.

Intramuscular administration to the thigh or the upper arm is preferred. Injection may be via a needle (e.g., a hypodermic needle), but needle-free injection may alternatively be used. A typical intramuscular dose is 0.5 ml.

The compositions disclosed herein may be used to elicit systemic and/or mucosal immunity.

Dosage treatment can be a single dose schedule or a multiple dose schedule. Multiple doses may be used in a primary immunization schedule and/or in a booster immunization schedule. A primary dose schedule may be followed by a booster dose schedule. Suitable timing between priming doses (e.g., between 4-16 weeks), and between priming and boosting, can be routinely determined.

Bacterial infections affect various areas of the body and so compositions may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition). The composition may be prepared for topical administration, e.g., as an ointment, cream or powder. The composition be prepared for oral administration, e.g., as a tablet or capsule, or as a syrup (optionally flavored). The composition may be prepared for pulmonary administration, e.g. as an inhaler, using a fine powder or a spray. The composition may be prepared as a suppository or pessary. The composition may be prepared for nasal, aural or ocular administration, e.g. as spray, drops, gel or powder.

Screening Methods

This disclosure provides a process for determining whether a test compound binds to a polypeptide disclosed herein. If a test compound binds to a polypeptide disclosed herein and this binding inhibits the life cycle or the infectivity of the pathogenic bacteria, then the test compound can be used as an antibiotic or as a lead compound for the design of antibiotics. The process will typically comprise the steps of contacting a test compound with a polypeptide disclosed herein, and determining whether the test compound binds to said polypeptide. Suitable test compounds include polypeptides, polypeptides, carbohydrates, lipids, nucleic acids (e.g., DNA, RNA, and modified forms thereof), as well as small organic compounds (e.g., MW between 200 and 2000 Da). The test compounds may be provided individually, but will typically be part of a library (e.g., a combinatorial library). Methods for detecting a binding interaction include NM1R, filter-binding assays, gel-retardation assays, displacement assays, surface plasmon resonance, reverse two-hybrid, etc. A compound which binds to a polypeptide of the invention can be tested for antibiotic or anti-infective activity by contacting the compound with bacteria and then monitoring for inhibition of growth or inability to infect host cells. This disclosure also includes compounds identified using these methods.

Preferably, the process comprises the steps of: (a) contacting a polypeptide disclosed herein with one or more candidate compounds to give a mixture; (b) incubating the mixture to allow polypeptide and the candidate compound(s) to interact; and (c) assessing whether the candidate compound binds to the polypeptide or modulates its activity.

Once a candidate compound has been identified in vitro as a compound that binds to a polypeptide disclosed herein then it may be desirable to perform further experiments to confirm the in vivo function of the compound in inhibiting bacterial growth and/or survival. Thus the method comprises the further step of contacting the compound with a pathogenic bacterium and assessing its effect.

The polypeptide used in the screening process may be free in solution, affixed to a solid support, located on a cell surface or located intracellularly. Preferably, the binding of a candidate compound to the polypeptide is detected by means of a label directly or indirectly associated with the candidate compound. The label may be a fluorophore, radioisotope, or other detectable label.

The use and practice of the disclosed polypeptides, nucleic acids and antibodies will employ, unless otherwise indicated, conventional methods of chemistry, biochemistry, molecular biology, immunology and pharmacology, within the skill of the art. Such techniques are explained fully in the literature.

### REFERENCES

The following references and the references found throughout are hereby incorporated by reference for their teachings and in particular for the purpose and teaching specifically referenced herein.
1. Dayhoff, M. O. (1969) Sci Am 221, 86-95.
2. Dayhoff, M. O. (1976) Fed Proc 35, 2132-8.
3. Tatusov, R. L., Koonin, E. V. & Lipman, D. J. (1997) Science 278, 631-7.
4. Hacker, J. & Kaper, J. B. (2000) Annual Review of Microbiology 54, 641-679.
5. Feil, E. J. (2004) Nat Rev Microbiol 2, 483-95.
6. Doolittle, W. F. (1999) Science 284, 2124-2128.
7. Galan, J. E. & Collmer, A. (1999) Science 284, 1322-8.
8. Blocker, A., Komoriya, K. & Aizawa, S. (2003) Proc Natl Acad Sci USA 100, 3027-30.
9. Hueck, C. J. (1998) Microbiol Mol Biol Rev 62, 379-433.
10. Macnab, R. M. (1999) J Bacteriol 181, 7149-53.
11. Gophna, U., Ron, E. Z. & Graur, D. (2003) Gene 312, 151-163.
12. Covacci, A., Telford, J. L., Del Giudice, G., Parsonnet, J. & Rappuoli, R. (1999) Science 284, 1328-33.
13. Christie, P. J. (2001) Mol Microbiol 40, 294-305.
14. Cascales, E. & Christie, P. J. (2003) Nat Rev Microbiol 1, 137-49.
15. Albert, R. & Barabasi, A. L. (2002) Reviews of Modern Physics 74, 47-97.
16. Erdös, P. & Rényi, A. (1959) Publ. Math. (Debrecen) 6, 290-291.
17. Ravasz, E., Somera, A. L., Mongru, D. A., Oltvai, Z. N. & Barabasi, A. L. (2002) Science 297, 1551-5.
18. Newman, M. E. J. (2003) Siam Review 45, 167-256.
19. Newman, M. E. J. & Girvan, M. (2004) Physical Review E 69, 26113-26127.
20. Bateman, A., Coin, L., Durbin, R., Finn, R. D., Hollich, V., Griffiths-Jones, S., Khanna, A., Marshall, M., Moxon, S., Sonnhammer, E. L., Studholme, D. J., Yeats, C. & Eddy, S. R. (2004) Nucleic Acids Res 32 Database issue, D138-41.
21. Altschul, S. F., Madden, T. L., Schaffer, A. A., Zhang, J., Zhang, Z., Miller, W. & Lipman, D. J. (1997) Nucleic Acids Res 25, 3389-402.
22. Albert, R. and Barabasi, A.-L. (2002) Rev. Mod. Phys 74, 47-97.
23. Watanabe, H. and Otsuka, J. (1995) Comput. Appl. Biosci. 11, 159-166.
24. Teichmann, S. A., Park, J. and Chothia, C. (1998) Proc. Nat. Acad. Sci. USA 95, 14658-14663.
25. Koonin, E. V., Yuri, I. W. and Karev, G. P. (2002) Nature (London) 420, 218-223.
26. Spang, R. and Vingron, M. (2001) Bioinformatics 17, 338-342.
27. Ravasz, E., Somera, A. L., Mongru, D. A., Oltvai, Z. N. and Barabasi, A.-L. (2002) Science 297, 1551-1555.
28. Spirin, V. and Mirny, L. A. (2003) Proc. Natl. Acad. Sci. USA 100, 12123-12128.
29. Jeong, H., Tombor, B., Albert A., Oltvai, Z. N., and Barabasi, A.-L. (2000) Nature (London) 407, 651-654.
30. Joeng, H., Mason, S. P., Barabasi, A.-L., and Oltvai, Z. N. (2001) Nature (London) 411, 41-42.
31. Dokholyan, N. V., Shaknovich, B. and Shakhnovich, E. I. (2002) Proc. Natl. Acad. Sci. USA 99, 14132-14136.
32. Arita, M. (2004) Proc. Natl. Acad. Sci. USA 101, 1543-1547.
33. Albert, R., Jeong, H. and Barabasi, A.-L. (1999) Nature (London) 401, 130-131.
34. Hubermann, B. A., Pirolli, P. L. T., Pitkow, J. E. and Lukose, R. M. (1998) Science 280, 95-97.
35. Babarasi, A.-L., Jeong, H., Neda, Z., Ravasz, E., Schubert, A. and Vicsek, T. (2001) Physica A 311, 590-614.
36. Newman, M. E. J. (2001) Phys. Rev. E 64, 16131-16138.
37. Smith and Waterman (1981) Adv. Appl. Math. 2:482.
38. Needleman and Wunsch (1970) J. Mol. Biol. 48:443.
39. Pearson and Lipman (1988) Proc. Natl. Acad. Sci. USA 85:2444.
40. Devereux et al. (1984) Nucl. Acid Res. 12:387-395.
41. Feng and Doolittle (1987) J. Mol. Evol. 35:351-360.
42. Higgins and Sharp (1989) CABIOS 5:151-153.
43. Altschul et al. (1990) J. Mol. Biol. 215:403-410.
44. Karlin et al. (1993) Proc. Natl. Acad. Sci. USA 90:5873-5787.
45. Altschul et al. (1996) Methods in Enzymology 266: 460-480.
46. Thompson, J. D., Higgins, D. G. & Gibson, T. J. (1994) Nucleic Acids Res 22: 4673-80.
47. Felsenstein, J. (2005) PHYLIP Phylogeny Inference Package) version 3.6. Distributed by the author. Department of Genome Sciences, University of Washington, Seattle.
48. Kaufman, L. and Rousseeuw, P. J. (1990) Finding Groups in Data. An Introduction to Cluster Analysis. (Wiley, New York).
49. Barrat, A. and Weigt, M. (2000) Eur. Phys. J. B 13:547.
50. Felek et al. (2003) Infection and Immunity 71(10):6063-6067.
51. Nishikawa et al. (2006) J Clin Invest 116(7):1946-1954.

Lengthy table referenced here
US20090327170A1-20091231-T00001
Please refer to the end of the specification for access instructions.

Lengthy table referenced here
US20090327170A1-20091231-T00002
Please refer to the end of the specification for access instructions.

Lengthy table referenced here
US20090327170A1-20091231-T00003
Please refer to the end of the specification for access instructions.

Lengthy table referenced here
US20090327170A1-20091231-T00004
Please refer to the end of the specification for access instructions.

Lengthy table referenced here
US20090327170A1-20091231-T00005
Please refer to the end of the specification for access instructions.

Lengthy table referenced here
US20090327170A1-20091231-T00006
Please refer to the end of the specification for access instructions.

Lengthy table referenced here
US20090327170A1-20091231-T00007
Please refer to the end of the specification for access instructions.

Lengthy table referenced here
US20090327170A1-20091231-T00008
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20090327170A1). An electronic copy of the table will also be available from the USPTO upon request and payment of the fee set forth in 37 CFR 1.19(b)(3).

What we claim is:

1. A method for generating a sequence similarity network comprising one or more sequence similarity families within a dataset of sequences comprising:
(a) providing a sequence similarity network generated from the dataset of sequences, wherein each node in the sequence similarity network represents a sequence from the dataset and each pair of nodes is connected by a link which meets a sequence similarity criterion; and
(b) rewiring the sequence similarity network by applying an overlap criterion to at least one pair of nodes.

2. The method of claim 1 wherein the overlap criterion includes removing the link between the pair of nodes if the overlap criterion is not met.

3. The method of claim 2 wherein the rewiring removes at least fifty percent false links.

4. The method of claim 2 wherein the rewiring removes at least sixty percent false links.

5. The method of claim 2 wherein the rewiring removes at least seventy percent false links.

6. The method of claim 2 wherein the overlap criterion includes adding the link between the nodes pair of nodes if the overlap criterion is met.

7. The method of claim 3 wherein the rewiring adds fewer than fifty percent false links.

8. The method of claim 3 wherein the rewiring adds fewer than forty percent false links.

9. The method of claim 3 wherein the rewiring adds fewer than thirty percent false links.

10. The method of claim 3 wherein the overlap criterion is met when an overlap coefficient for a pair of sequences is greater than or equal to an overlap threshold.

11. The method of claim 10 wherein the overlap threshold is determined by:
(c) determining the connectivity coefficient for each sequence similarity network generated by performing steps (a) and (b) for a set of overlap thresholds; and
(d) selecting an overlap threshold from the set of overlap thresholds that yields a modularity coefficient of at least about 0.3.

12. The method of claim 11 wherein the selected overlap threshold yields a modularity coefficient of at least about 0.5.

13. The method of claim 11 wherein the selected overlap threshold yields a modularity coefficient of at least about 0.6.

14. The method of claim 11 wherein the selected overlap threshold yields a modularity coefficient of at least about 0.7.

15. The method of claim 11 wherein the selected overlap threshold yields the highest modularity coefficient.

16. The method of claim 10 wherein the overlap threshold is between about 0.4 and about 0.6.

17. The method of claim 10 wherein the overlap threshold is about 0.5.

18. The method of claim 1 wherein the sequence similarity criterion is met when the sequence similarity index for a pair of sequences indicates similarity more significant than a sequence similarity threshold.

19. The method of claim 16 wherein the sequence similarity threshold is an E-value of 10⁻¹.

20. The method of claim 1 further comprising the step of identifying a sequence similarity family within the rewired sequence similarity network that includes a sequence of interest.

21. The method of claim 20 wherein the sequence of interest is selected from the group of sequences comprising an antigenic protein sequence, an antibody therapeutic target protein sequence, and a small molecule therapeutic target protein sequence.

22. A method for annotating sequences within a dataset of sequences comprising:
(a) providing a dataset of sequences comprising one or more annotated sequences and one or more unannotated sequences;
(b) providing a sequence similarity network generated from the dataset of sequences, wherein each node in the sequence similarity network represents a sequence from the dataset and each pair of nodes is connected by a link which meets a sequence similarity criterion; and
(c) partitioning the sequence similarity network into sequence similarity families by applying an overlap criterion to at least one pair of nodes; and
(d) annotating the one or more unannotated sequences by identifying a sequence similarity family that includes at least one unannotated sequence and adding an annotation to the at least one unannotated sequence based upon at least one annotated sequence in the sequence similarity family.

23. A method for identifying an evolutionarily-related family of sequences within a dataset of sequences comprising:
(a) providing a sequence similarity network generated from the dataset of sequences, wherein each node in the sequence similarity network represents a sequence from the dataset and each pair of nodes is connected by a link which meets a sequence similarity criterion; and
(c) partitioning the sequence similarity network into sequence similarity families by applying an overlap criterion to at least one pair of nodes; and
(d) identifying at least one sequence similarity family as an evolutionarily-related family.

24. The method of claim 23 wherein the partitioning removes at least one sequence from the sequence similarity family that is not evolutionarily related to the sequences in the sequence similarity family, but has greater homology at the primary sequence level to at least one sequence in the sequence similarity family than between at least one pair of sequences in the sequence similarity family.

25. A method for annotating sequences within a dataset of sequences comprising:
(a) providing a dataset of sequences comprising one or more annotated sequences and one or more unannotated sequences;
(b) providing a sequence similarity network generated from the dataset of sequences, wherein each node in the sequence similarity network represents a sequence from the dataset and each pair of nodes is connected by a link which meets a sequence similarity criterion; and
(c) partitioning the sequence similarity network into sequence similarity families by applying an overlap criterion to at least one pair of nodes; and
(e) annotating the one or more unannotated sequences by identifying a sequence similarity family that includes at least one unannotated sequence and adding an annotation to the at least one unannotated sequence based upon at least one annotated sequence in the sequence similarity family.

26. A computer-readable medium having computer-executable instructions for performing a method of a sequence similarity network comprising one or more sequence similarity families within a dataset of sequences, the method comprising:
(a) providing a sequence similarity network generated from the dataset of sequences, wherein each node in the sequence similarity network represents a sequence from the dataset and each pair of nodes is connected by a link which meets a sequence similarity criterion; and
(b) rewiring the sequence similarity network by applying an overlap criterion to at least one pair of nodes.

27. A computerized system for performing a method of a sequence similarity network comprising one or more sequence similarity families within a dataset of sequences, the system comprising:
means for providing a sequence similarity network generated from the dataset of sequences, wherein each node in the sequence similarity network represents a sequence from the dataset and each pair of nodes is connected by a link which meets a sequence similarity criterion; and
means for rewiring the sequence similarity network by applying an overlap criterion to at least one pair of nodes.

28. A computerized system comprising a computer-readable medium containing a sequence similarity network comprising one or more sequence similarity families.

29. An isolated polypeptide comprising an amino acid sequence which has at least 75% sequence identity to an amino acid sequence selected from the group consisting of SEQ ID NOS:1-1284.

30. The polypeptide of claim 30, wherein the amino acid sequence is selected from the group consisting of SEQ ID NOS:1-1284.

31. An isolated polypeptide comprising a fragment of at least 7 consecutive amino acids from an amino acid sequence selected from the group consisting of SEQ ID NOS:1-1284.

32. The polypeptide of claim 31, wherein the fragment comprises a T-cell or a B-cell epitope from an amino acid sequence selected from the group consisting of SEQ ID NOS:1-1284.

33. An antibody which binds to a polypeptide selected from:
(a) a polypeptide comprising an amino acid sequence which has at least 75% sequence identity to an amino acid sequence selected from the group consisting of SEQ ID NOS:1-1284;
(b) a polypeptide comprising an amino acid sequence selected from the group consisting of SEQ ID NOS:1-1284;
(c) a polypeptide comprising a fragment of at least 7 consecutive amino acids from an amino acid sequence selected from the group consisting of SEQ ID NOS:1-1284; and
(d) a polypeptide comprising a fragment of at least 7 consecutive amino acids, wherein the fragment comprises a T-cell or a B-cell epitope from an amino acid sequence selected from the group consisting of SEQ ID NOS:1-1284.

34. The antibody of claim 33 which is monoclonal.

35. An isolated nucleic acid comprising a nucleotide sequence which encodes an amino acid sequence that has at least 75% sequence identity to an amino acid sequence selected from the group consisting of SEQ ID NOS:1-1284.

36. The nucleic acid of claim 35, comprising a nucleotide sequence which encodes an amino acid sequence selected from the group consisting of SEQ ID NOS: 1-1284.

37. An isolated nucleic acid which can hybridize under high stringency conditions to a nucleotide sequence which encodes an amino acid sequence selected from the group consisting of SEQ ID NOS: 1-1284.

38. An isolated nucleic acid comprising a fragment of 10 or more consecutive nucleotides from a nucleotide sequence which encodes an amino acid sequence selected from the group consisting of SEQ ID NOS: 1-1284.

39. An isolated nucleic acid encoding the polypeptide of selected from the group comprising:
(a) a polypeptide comprising an amino acid sequence which has at least 75% sequence identity to an amino acid sequence selected from the group consisting of SEQ ID NOS:1-1284;
(b) a polypeptide comprising an amino acid sequence selected from the group consisting of SEQ ID NOS:1-1284;
(c) a polypeptide comprising a fragment of at least 7 consecutive amino acids from an amino acid sequence selected from the group consisting of SEQ ID NOS:1-1284; and
(d) a polypeptide comprising a fragment of at least 7 consecutive amino acids, wherein the fragment comprises a T-cell or a B-cell epitope from an amino acid sequence selected from the group consisting of SEQ ID NOS:1-1284.

40. A composition comprising: (a) the polypeptide according to claims 29, 30, 31, or 32, the antibody according to claim 33, or the nucleic acid according to claim 39; and (b) a pharmaceutically acceptable carrier.

41. The composition of claim 40, further comprising a vaccine adjuvant.

42. The composition of claim 40 for use as a medicament.

43. A method of treating a patient, comprising administering to the patient a therapeutically effective amount of the composition of claim 40.

44. Use of the composition of claim 40 in the manufacture of a medicament for treating or preventing disease and/or infection caused by the pathogenic bacteria from which the composition was derived.
